SEQUENCE LISTING (62 sequences)

SEQ ID NO: 1 - protein, 5 aa, synthetic construct
DYGVH

SEQ ID NO: 2 - protein, 16 aa, synthetic construct
VIWAGGGTNYNSALMS

SEQ ID NO: 3 - protein, 12 aa, synthetic construct
DKGYSYYYSMDY

SEQ ID NO: 4 - protein, 15 aa, synthetic construct
RASESVEYYVTSLMQ

SEQ ID NO: 5 - protein, 7 aa, synthetic construct
AASNVES

SEQ ID NO: 6 - protein, 9 aa, synthetic construct
QQSRKVPYT

SEQ ID NO: 7 - protein, 9 aa, synthetic construct
RMFPNAPYL

SEQ ID NO: 8 - protein, 9 aa, synthetic construct
CMTWNQMNL

SEQ ID NO: 9 - protein, 9 aa, synthetic construct
VLDFAPPGA

SEQ ID NO: 10 - protein, 9 aa, synthetic construct
SLGEQQYSV

SEQ ID NO: 11 - protein, 9 aa, synthetic construct
VLDGLDVLL

SEQ ID NO: 12 - protein, 9 aa, synthetic construct
NLTHVLYPV

SEQ ID NO: 13 - protein, 10 aa, synthetic construct
SLYSFPEPEA

SEQ ID NO: 14 - protein, 10 aa, synthetic construct
ALYVDSLFFL

SEQ ID NO: 15 - protein, 9 aa, synthetic construct
SLLQHLIGL

SEQ ID NO: 16 - protein, 10 aa, synthetic construct
ELTLGEFLKL

SEQ ID NO: 17 - protein, 10 aa, synthetic construct
LDRERAKNKI

SEQ ID NO: 18 - protein, 9 aa, synthetic construct
FLDRFLSCM

SEQ ID NO: 19 - protein, 11 aa, synthetic construct
SLIAAAAFCLA

SEQ ID NO: 20 - protein, 9 aa, synthetic construct
YISPWILAV

SEQ ID NO: 21 - protein, 9 aa, synthetic construct
YLFPQLISV

SEQ ID NO: 22 - protein, 9 aa, synthetic construct
GLVGLIFAV

SEQ ID NO: 23 - protein, 9 aa, synthetic construct
SLFVLGLFL

SEQ ID NO: 24 - protein, 9 aa, synthetic construct
SLLMWITQA

SEQ ID NO: 25 - protein, 10 aa, synthetic construct
ALSPASSRSV

SEQ ID NO: 26 - protein, 9 aa, synthetic construct
CLGGLLTMV

SEQ ID NO: 27 - protein, 9 aa, synthetic construct
FLYALALLL

SEQ ID NO: 28 - protein, 9 aa, synthetic construct
GLCTLVAML

SEQ ID NO: 29 - protein, 9 aa, synthetic construct
YVLDHLIVV

SEQ ID NO: 30 - protein, 10 aa, synthetic construct
LLDFVRFMGV

SEQ ID NO: 31 - protein, 9 aa, synthetic construct
YLQQNWWTL

SEQ ID NO: 32 - protein, 9 aa, synthetic construct
IYVLVMLVL

SEQ ID NO: 33 - protein, 9 aa, synthetic construct
TYPVLEEMF

SEQ ID NO: 34 - protein, 9 aa, synthetic construct
DYNFVKQLF

SEQ ID NO: 35 - protein, 9 aa, synthetic construct
RYSIFFDYM

SEQ ID NO: 36 - protein, 9 aa, synthetic construct
TYSAGIVQI

SEQ ID NO: 37 - protein, 9 aa, synthetic construct
RPPIFIRRL

SEQ ID NO: 38 - protein, 9 aa, synthetic construct
QPRAPIRPI

SEQ ID NO: 39 - protein, 13 aa, synthetic construct
RPQGGSRPEFVKL

SEQ ID NO: 40 - protein, 9 aa, synthetic construct
GILGFVFTL

SEQ ID NO: 41 - protein, 9 aa, synthetic construct
QPEWFRNVL

SEQ ID NO: 42 - protein, 9 aa, synthetic construct
SPIVPSFDM

SEQ ID NO: 43 - protein, 9 aa, synthetic construct
QYDPVAALF

SEQ ID NO: 44 - protein, 9 aa, synthetic construct
VYALPLKML

SEQ ID NO: 45 - protein, 9 aa, synthetic construct
AYAQKIFKI

SEQ ID NO: 46 - protein, 10 aa, synthetic construct
TPRVTGGGAM

SEQ ID NO: 47 - protein, 11 aa, synthetic construct
RPHERNGFTVL

SEQ ID NO: 48 - protein, 9 aa, synthetic construct
ILSLELMKL

SEQ ID NO: 49 - protein, 9 aa, synthetic construct
SLEENIVIL

SEQ ID NO: 50 - protein, 9 aa, synthetic construct
KLLEYIEEI

SEQ ID NO: 51 - protein, 9 aa, synthetic construct
KLQEELNKV

SEQ ID NO: 52 - protein, 9 aa, synthetic construct
KLKGKEAEL

SEQ ID NO: 53 - protein, 9 aa, synthetic construct
VLQELNVTV

SEQ ID NO: 54 - protein, 10 aa, synthetic construct
ELAGIGILTV

SEQ ID NO: 55 - protein, 9 aa, synthetic construct
IMDQVPFSV

SEQ ID NO: 56 - protein, 9 aa, synthetic construct
SLLMWITQC

SEQ ID NO: 57 - protein, 9 aa, synthetic construct
SLLMWITQA

SEQ ID NO: 58 - protein, 9 aa, synthetic construct
NLVPMVATV

SEQ ID NO: 59 - protein, 9 aa, synthetic construct
ISPWILAVL

SEQ ID NO: 60 - protein, 8 aa, synthetic construct
VYPEGSSL

SEQ ID NO: 61 - protein, 9 aa, synthetic construct
IFAVCLVGF

SEQ ID NO: 62 - protein, 9 aa, synthetic construct
LFVLGLFLW

(19) United States
(12) Patent Application Publication
(10) Pub. No.: US 2026/0174799 A1
(43) Pub. Date: Jun. 25, 2026

(54) COMBINATION THERAPY WITH T CELL ENGAGER

(71) Applicant: CellKure Inc., Newport Coast, CA (US)

(72) Inventors: Daniel Bednarik, Gaithersburg, MD (US); Mathias Oelke, Gaithersburg, MD (US); Jerome B. Zeldis, Gaithersburg, MD (US); Ruipeng Wang, Gaithersburg, MD (US); Jack Ragheb, Gaithersburg, MD (US); Adam Parks, Gaithersburg, MD (US)

(21) Appl. No.: 19/260,821

(22) Filed: Jul. 7, 2025

Related U.S. Application Data
(63) Continuation of application No. PCT/US2024/010409, filed on Jan. 5, 2024.
(60) Provisional application No. 63/437,153, filed on Jan. 5, 2023.
(60) Provisional application No. 63/444,696, filed on Feb. 10, 2023.

(51) Int. Cl. A61K 35/17; A61K 39/00; A61K 40/11; A61K 40/33; A61P 35/02; C07K 16/28

(52) U.S. Cl. CPC A61K 35/17; A61K 40/11; A61K 40/33; A61P 35/02; C07K 16/2803; C07K 16/2809; C07K 16/2866; C07K 16/2878; A61K 2039/505; A61K 2239/38; A61K 2239/48; C07K 2317/31; C07K 2317/622

(57) ABSTRACT

In the various aspects and embodiments the present disclosure provides for potentiation of cytotoxic T cell (CTL) therapies with T cell engagers. Cytotoxic T cell therapies, including CAR-T therapies and CTL therapies based on natural TCR repertoires, often require high effector to target ratio for effective killing. However, this ratio is substantially reduced according to the present disclosure by co-administering T cell engager therapy (e.g., BiTE therapy). Further, by co-administering a T cell therapy with T cell engager therapy, the therapeutic window for the T cell engager therapy is substantially improved.

### PRIORITY

This Application is a National Phase Application under 35 U.S.C. § 371 of International Application No. PCT/US2024/010409, filed Jan. 5, 2024, which claims priority to, and the benefit of, U.S. provisional application No. 63/437,153 filed Jan. 5, 2023 and provisional application No. 63/444,696 filed Feb. 10, 2023.

### DESCRIPTION OF THE TEXT FILE SUBMITTED ELECTRONICALLY

The instant application contains a sequence listing, which has been submitted in XML format via EFS-Web. The contents of the XML copy named “NEX-015PC_Sequence_Listing.xml,” which was created on Jul. 7, 2025 and is 54,685 bytes in size, the contents of which are incorporated herein by reference in their entirety.

### BACKGROUND

While immunotherapies, including immune checkpoint inhibitors, immune cell therapies (including CAR-T cells), and various antibodies and bispecific antibodies specific for immune and tumor targets, have yielded good results in a minority of cancer patients, non-responsiveness or resistance to therapy, partial responses, off-target toxicity, and disease recurrence remain significant challenges. Further improvements in immunotherapy are therefore needed including for cancer treatment. In the various aspects and embodiments, the present disclosure meets these objectives.

### DESCRIPTION OF THE FIGURES

FIG. 1A and FIG. 1B are graphs of a cytotoxicity assay of effector cells (E) mixed with target cells (T) (U266 multiple myeloma cells) at the listed effector:target (E:T) ratios. FIG. 1A shows results with various effector cells including CD4+ T cells, CD8+ T cells, Epstein-Barr virus (EBV)-specific cytotoxic T lymphocytes (CTLs), and multiple myeloma (MM; red, closed squares) specific CTLs mixed with target U266 cells at the listed E:T ratios. FIG. 1B shows results with CD4+ T cells, CD8+ T cells, Epstein-Barr virus (EBV)-specific cytotoxic T lymphocytes (CTLs), and multiple myeloma (MM; red, closed squares) specific CTLs mixed with a bispecific T cell engager (BiTE) and target U266 cells at the listed E:T ratios.

FIG. 2A and FIG. 2B are graphs of a cytotoxicity assay of effector cells (E) mixed with target cells (T) from a multiple myeloma cell line (MM.1S cells) at the listed E:T ratios. FIG. 2A shows results with CD4+ T cells, CD8+ T cells, Epstein-Barr virus (EBV)-specific cytotoxic T lymphocytes (CTLs), and multiple myeloma specific CTLs mixed with target MM.1S cells at the listed E:T ratios. FIG. 2B shows results with the same effector cells mixed with a bispecific T cell engager (CD3×BCMA BITE) and target MM.1S cells at the listed E:T ratios.

FIG. 3 shows results using an EBV-infected cell line (LAZ) as target cell. EBV-specific CTLs produced ex vivo using the Enrichment+Expansion (E+E) process were substantially potentiated by CD3×BCMA BiTE at 0.2 μM. Uneducated CD8+ cells produce low killing. At 5:1 E:T ratio E+E cells (with 0.2 μM BiTE) produced about 75% killing. Without BiTE, neither cells produced appreciable killing at 2.5:1 E:T ratio.

FIG. 4 is an image showing example BiTE molecules schematically: αFlt3-αCD3; αCD123-αCD3; αCD33-αCD3; and αSIGLEC6-αCD3.

FIG. 5A and FIG. 5B are graphs showing a dose titration of αFlt3 BiTE killing of HLA-A2⁺ acute myeloid leukemia (AML) target cells. FIG. 5A shows a dose titration of αFlt3 BiTE killing of OCI-AML-2 AML cell line. FIG. 5B shows a dose titration of αFlt3 BiTE killing of OCI-AML-3 AML cell line.

FIG. 6A and FIG. 6B are graphs showing a dose titration of αFlt3 BiTE killing of HLA-A2⁺ AML target cells. FIG. 6A shows a dose titration of αFlt3 BiTE killing of AML14 AML cell line. FIG. 6B shows a dose titration of αFlt3 BiTE killing of THP1 AML cell line.

FIG. 7A and FIG. 7B are graphs showing a dose titration of αCD123 BiTE killing of HLA-A2⁺ AML target cells. FIG. 7A shows a dose titration of αCD123 BiTE killing of AML14 AML cell line. FIG. 7B shows a dose titration of αCD123 BiTE killing of THP1 AML cell line.

FIG. 8A and FIG. 8B are graphs showing a dose titration of αCD133 BiTE killing of HLA-A2⁺ acute myeloid leukemia (AML) target cells. FIG. 8A shows a dose titration of αCD133 BiTE killing of OCI-AML-2 AML cell line. FIG. 8B shows a dose titration of αCD133 BiTE killing of OCI-AML-3 AML cell line.

FIG. 9A and FIG. 9B are graphs showing a dose titration of αCD133 BiTE killing of HLA-A2⁺ AML target cells. FIG. 13A shows a dose titration of αCD133 BiTE killing of AML14 AML cell line. FIG. 9B shows a dose titration of αCD133 BiTE killing of THP1 AML cell line.

FIG. 10A and FIG. 10B are graphs showing a dose titration of αFlt3 BiTE killing of HLA-A2⁺ AML target cells. FIG. 10A shows a dose titration of αFlt3 BiTE killing of AML14 AML cell line. FIG. 10B shows a dose titration of αFlt3 BiTE killing of THP1 AML cell line.

FIG. 11 illustrates a bimodal nanoparticle that allows for activation of antigen-specific T cells, followed by delivery of mRNA encoding the BiTE to the T cell.

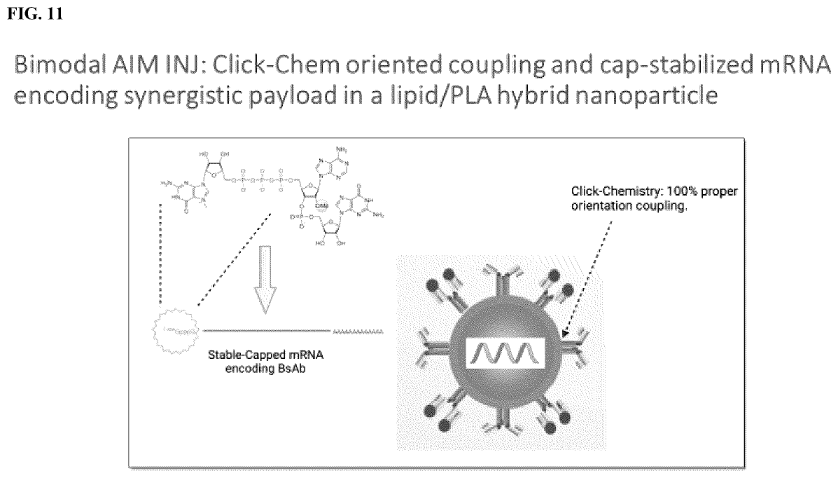

FIG. 12A and FIG. 12B show that artificial immune modulation (AIM) CTL prepared according to this disclosure act synergistically with T cell engagers (TCE). FIG. 12A compares AML-specific AIM CTL with polyclonal CTL for in vitro killing of HLA-A2+ MOLM-13 cells. As shown, polyclonal CTL show little TCR-mediated killing, while AIM CTL have a modest effect, at the subtherapeutic effector:target ratio of 0.25:1. As shown in FIG. 12B, with the addition of a CD123×CD3 bispecific antibody, killing by AIM CTL was substantial at the same E:T ratio (reaching about 90% at 72 hours), while TCR-mediated killing by polyclonal CTL remained insignificant.

### DETAILED DESCRIPTION

In various aspects and embodiments, the present disclosure provides for potentiation of cytotoxic T cell (CTL) therapies with T cell engagers. As demonstrated herein, cytotoxic T cell therapies, including CAR-T therapies and CTL therapies based on natural TCR repertoires, often require high effector to target ratio for effective killing. However, this ratio is substantially reduced according to the present disclosure by co-administering T cell engager therapy (referred to herein as BiTE therapy or TCE therapy). Further, by co-administering a T cell therapy with T cell engager therapy, the therapeutic window for the T cell engager therapy is substantially improved.

In aspects and embodiments, the present disclosure provides a method for treating a subject in need of a T cell therapy. In various embodiments, the method comprises administering a cytotoxic T lymphocyte (CTL) therapy to the subject, and administering one or more bispecific T cell engagers (BiTE). In various embodiments, the BiTE is administered at an amount effective to potentiate the CTL therapy. BiTEs can be administered as a composition comprising the BiTE polypeptide or complex, or as a polynucleotide encoding the BiTE, such as an mRNA. mRNA delivery, such as with lipid nanoparticles, is described elsewhere herein. In some embodiments, when delivering T cells activated and/or expanded ex vivo, a BiTE expressing polynucleotide (e.g., mRNA) may be introduced into the cells ex vivo.

T cell engager (TCE) molecules allow for a targeted immunotherapy and engagement with a patient's own T cells to treat hematologic malignancies and solid tumors. Since the approval of the CD19-targeted BiTE (bispecific T-cell engager) molecule blinatumomab, multiple TCE molecules against different targets have been developed in several tumor types. The terms TCE and BiTE are used interchangeably herein. Despite the promise of BiTE molecules, challenges remain, such as the need for continuous intravenous administration and low productivity, as well as adverse events such as cytokine release syndrome. In accordance with aspects of this disclosure, productivity and safety of BiTE therapies is improved by the co-therapies described herein.

In aspects and embodiments, the present disclosure provides BiTEs for combination therapy in numerous types of cancers and other conditions where CTL therapy is of need, and such BiTEs can be selected according to the particular disease. For example, BiTEs disclosed herein can engage with T cells through binding to a T-cell receptor (TCR) or component thereof, such as CD3, and engage a target cell through a tumor-associated or viral-associated antigen. In some embodiments, the BiTE activates T cells through binding to CD3. Exemplary tumor-associated antigens include but are not limited to CD19, EpCAM, Her2, CD20, CD123, and BCMA (among others described herein). Concurrent binding to both the T cell target (e.g., CD3) and the tumor- or viral-associated antigen results in potent lysing of the target cell. Various molecular constructs for BiTEs, including bispecific antibodies, dimerized natural ligands, and DARTs, are disclosed herein.

In various embodiments, the CTLs express a chimeric antigen receptor (CAR) or a recombinant TCR, including against any tumor antigen described herein. Exemplary CARs can target CD33, CD19, CD7, CD123, or CD371. Exemplary CAR-T therapies include Tisagenlecleucel, also known as tisa-cel (KYMRIAH), Axicabtagene ciloleucel, also known as axi-cel (YESCARTA), Brexucabtagene autoleucel, also known as brexu-cel (TECARTUS), Lisocabtagene maraleucel, also known as liso-cel (BREYANZI), Idecabtagene vicleucel, also known as ide-cel (ABECMA), and Ciltacabtegene autoleucel, also known as cilta-cel (CARVYKTI). Other CAR-T cells can target antigens such as: Human epidermal growth factor receptor 2 (HER2) (e.g., for ovarian cancer, breast cancer, glioblastoma, colon cancer, osteosarcoma, and medulloblastoma); Epidermal growth factor receptor (EGFR) (e.g., for non-small cell lung cancer, epithelial carcinoma, and glioma); Mesothelin (e.g., for mesothelioma, ovarian cancer, and pancreatic adenocarcinoma); Prostate-specific membrane antigen (PSMA) (e.g., for prostate cancer); Carcinoembryonic antigen (CEA) (e.g., for pancreatic adenocarcinoma, breast cancer, and colorectal carcinoma); Glypican-3 (e.g., for hepatocellular carcinoma); Variant III of the epidermal growth factor receptor (EGFRvIII) (e.g., for glioblastoma); Disialoganglioside 2 (GD2) (e.g., for neuroblastoma and melanoma); Carbonic anhydrase IX (CAIX) (e.g., for renal cell carcinoma); Interleukin-13Ra2 (e.g., for glioma); Fibroblast activation protein (FAP) (e.g., for malignant pleural mesothelioma); L1 cell adhesion molecule (L1-CAM) (e.g., for neuroblastoma, melanoma, and ovarian); Cancer antigen 125 (CA 125) (e.g., for epithelial ovarian cancer); Cluster of differentiation 133 (CD 133) (e.g., for glioblastoma and cholangiocarcinoma, adenocarcinoma); Cancer/testis antigen 1B (CTAGIB) (e.g., for melanoma and ovarian cancer); Mucin 1 (e.g., for seminal vesicle cancer); Folate receptor-a (FR-a) (e.g., for ovarian cancer); and Growth factor receptors selected from one or more of ErbB1, ErbB2, ErbB3, or ErbB4, IGF1R, IGF2R, TβR I-II, VEGFR1, VEGFR2, VEGFR3, PDGFR (α/β), and FGFR1 through 4.

In some embodiments, the CTLs do not express a CAR or recombinant T cell receptor (TCR). For example, the CTLs can be activated and/or expanded ex vivo from peripheral blood or tumor infiltrating lymphocytes, optionally using artificial antigen presenting cells presenting one or more target antigens. While T cells in these embodiments can provide advantages in being specific for multiple antigens, and can provide advantages in persistence (by having a predominately memory phenotype), high effector to target ratio may be desired to drive patient response. As disclosed herein, BiTE co-therapy provides a surprising potentiation of the killing, and substantially reduces the effector to target ratio.

Accordingly, in some embodiments the CTLs comprise predominately a memory phenotype. In some embodiments, greater than about 50% of the CTLs comprise a memory phenotype; or greater than about 75%, or greater than about 85%, or greater than about 95% of the CTLs have a memory phenotype. Memory T cells include T memory stem cells (T_{SCM}), and central memory and effector memory T cells. Memory T cells are T cells that have previously responded to their cognate antigen. At a second encounter with the cognate antigen, memory T cells can reproduce to mount a faster and stronger immune response.

T memory stem cells (Tscm) are defined herein as CD45RA+ and as having at least the following surface markers: CD62L+, CD45RA+, and CD95+. In some embodiments, the T memory stem cells disclosed herein are CD62L+, CD45RA+, CD95+ and may have one or more of the following surface markers: CD28+, CD27+, CXCR3+CD11a+, IL-2Rβ+, CD58+, and CD57−. In some embodiments, the T memory stem cells comprise cells that are CD62L+, CD45RA+, CD28+, CD27+, and CD95+. In some embodiments, the T memory stem cells comprise cells that are CD62L+, CD45RA+, CD95+ and CXCR3+. In some embodiments, the T memory stem cells comprise cells that are CD62L+, CD45RA+, CD95+ and CD11a+. In some embodiments, the T memory stem cells comprise cells that are CD62L+, CD45RA+, CD95+ and IL-2Rβ+. In some embodiments, the T memory stem cells comprise cells that are CD62L+, CD45RA+, CD95+ and CD58+. In some embodiments, the T memory stem cells comprise cells that are CD62L+, CD45RA+, CD95+ and CD57−. This memory subpopulation has the stem cell-like capacity for self-renewal, as well as the multipotent capacity to reconstitute the memory and effector T cell subpopulations. T_{SCM} cells typically represent a small fraction of circulating T lymphocytes (e.g., >5%), and have the ability to proliferate rapidly and release inflammatory cytokines in response to antigen re-exposure. Accordingly, T_{SCM} cells are a subset of the memory T cell subpopulation. The T_{SCM} cells can be created and/or controlled using, as disclosed herein, an enrichment and expansion process with paramagnetic artificial Antigen Presenting Cells (aAPCs) and a recombinant T cell growth factor cocktail. In various embodiments, the CTL therapy comprises at least about 2%, or at least about 5%, or at least about 10% T_{SCM}.

In accordance with this disclosure, central memory T cells (T_{CM} cells) are defined herein as CD62L+ and CD45RA−. This memory subpopulation is commonly found in the lymph nodes and in the peripheral circulation. Effector memory T cells (TEM cells) are defined herein as CD62L− and CD45RA−. These memory T cells lack lymph node-homing receptors and are thus found in the peripheral circulation and tissues. T_{CM} cells display a capacity for self-renewal, and in accordance with embodiments of the invention, are also important for obtaining a long-lived effect. TEM cells also have some capacity for self-renewal, and strongly express genes essential to the cytotoxic function.

The CTL therapy does not comprise a large proportion of TEMRA cells. TEMRA stands for terminally differentiated effector memory cells re-expressing CD45RA (Temra). These cells do not have the capacity to divide, and are CD62L− and CD45RA+. TEMRA cells also provide robust cytotoxic function, but do not display a capacity for self-renewal.

In various aspects and embodiments, the CTL therapy comprises CTLs that are substantially composed of T_{SCM}, T_{CM} and TEM cells to balance duration of the effect versus destruction of the malignancy or other target cells. When combined with BiTE therapy, the cells also become potent killers of target cells with low effector to target ratio. For example, in some embodiments these cells make up at least about 75%, or at least about 80%, or at least about 90% of the T cells in the CTL therapy. In some embodiments, at least 50% of the CTLs have a central memory or effector memory phenotype, or at least 75% of the CTLs have a central memory or effector memory phenotype. In some embodiments, the CTLs are less than about 20% terminally differentiated effector memory cells (TEMRA), or less than about 15% TEMRA, or less than about 10% TEMRA, or less than about 5% TEMRA, or less than 2% TEMRA.

In various embodiments, the cell composition is at least 90% T cells, or at least 95% T cells, or at least 98%, or at least 99% T cells. For purposes of this disclosure, T cells are characterized by CD3+ cells. The T cells are generally CD8+. For example, the isolated cell composition may be characterized by having less than about 10%, or less than about 5% CD4+ T cells, or in some embodiments, less than about 2%, less than about 1.5%, or less than about 1% CD4+ T cells. When expanding CD8+ T cells ex vivo, CD4+ cells have a tendency to overgrow the CD8+ cells and compete for growth signals, and are not necessary for a robust and durable response. Thus, in embodiments CD4+ cells can be removed from the composition by positive selection of CD8+ cells or negative selection of CD4+ cells as is known in the art.

In some embodiments, at least 5% of the CTLs are specific for one or more target peptide antigens. In some embodiments, at least 10%, or at least 15%, or at least 20%, or at least 25%, or at least 30%, or at least 35%, or at least 40%, or at least 45%, or at least 50% of the CTLs are specific for one or more target peptide antigens. In some embodiments, less than about 75%, or less than about 50%, or less than about 25% of the CTLs are specific for one or more target peptide antigens (e.g., in the range of about 10% to about 40%, or 10% to about 30%, or 10% to about 25%). In various embodiments, the CTL therapy comprises CTLs specific for at least two target peptide antigens, or at least four target peptide antigens, or at least five target peptide antigen. In various embodiments, the CTL therapy contains T cells specific for from two to ten target peptide antigens, such as from two to six peptide antigens, or from four to ten peptide antigens (e.g., 4, 5, 6, 7, or 8 peptide antigens).

As used herein, the term “target peptide antigen(s)” or “target antigens” refers to peptide antigens employed ex vivo to enrich and/or expand the desired CD8+ cell population, for example in connection with artificial Antigen Presenting Cell (aAPC) or professional Antigen Presenting Cell (pAPC) platforms (e.g., dendritic cells). The aAPCs or pAPCs are employed to activate and expand CTLs from donor or patient lymphocytes. In some embodiments, the target peptide antigens are peptide epitopes loaded onto aAPCs for ex vivo enrichment and expansion of specific CD8+ T cells. Thus, the term “specific for the target peptide antigen” means that the T cell is antigen experienced with the target antigen. The term “target peptide antigen(s)” or “target antigens” can also refer to the specificity of a heterologous TCR. Exemplary target peptide antigens are disclosed elsewhere herein.

In various embodiments, the CTL therapy is as described in U.S. Pat. No. 10,987,412 or U.S. Pat. No. 11,007,222, which are hereby incorporated by reference in their entireties. For example, the CTLs can be prepared by ex vivo enrichment and expansion with paramagnetic aAPCs and peripheral blood, in a process referred to as enrichment or expansion (or E+E) or artificial immune modulation (AIM).

While expansion of CTLs ex vivo to therapeutic numbers in a short period of time (e.g., within about 2 weeks) can be challenging, the present invention in embodiments loosens the threshold for clinically-relevant numbers. In some embodiments, the CTLs are administered at a dose of from about 10⁶ to about 10¹⁰ cells per administration. In exemplary embodiments, the CTLs are administered at a dose of less than about 10⁹ cells per administration. For example, the CTLs are administered at a dose of less than about 10⁸ cells per administration, or less than about 5×10⁷ cells per administration, or less than about 10⁷ cells per administration. In some embodiments, the CTLs are administered at a dose of at least about 10⁷ cells or at least about 10⁸ cells per administration.

In other aspects, the disclosure provides a method for treating a subject in need of T cell therapy, where the method comprises administering an antigen presenting composition to the subject, thereby activating T cells in the subject in vivo. In this aspect, the invention further comprises administering one or more bispecific T cell engagers (BiTE) to potentiate the T cells activated in vivo. The BiTE can be administered in the same or different composition, including as a polynucleotide (such as an mRNA) encoding the BiTE. In accordance with these embodiments, the number of T cells activated and generated in vivo required for a therapeutic response is reduced by virtue of the BiTE co-therapy.

In various embodiments, the antigen presenting composition is a shelf-stable nano-aAPC composition for activating antigen-specific T cells in a patient. In addition to shelf-stable properties, the nanoscale aAPCs can be designed to provide pharmacodynamic advantages, including with respect to circulating properties, biodistribution, and degradation kinetics. These advantages result from physical parameters including particle size, surface charge, polydispersity index, polymer composition, ligand conjugation chemistry, ligand density, and peptide loading, among others. In some embodiments, the aAPCs persist in peripheral blood circulation sufficiently long to allow distribution to target tissues, including trafficking to lymphoid organs (e.g., lymph nodes) via blood/lymph exchange and/or trafficking to tumors and/or trafficking to target organs. In various embodiments, aAPCs can comprise inorganic core particles, such as gold or iron, and/or may be constructed of biocompatible lipids and/or polymers. In various embodiments, the aAPCs (e.g., polymeric aAPCs) can be as described in U.S. Pat. No. 10,632,193 or U.S. Pat. No. 11,510,981, each of which are hereby incorporated by reference in their entireties. aAPCs comprising inorganic cores include those described in U.S. Pat. Nos. 10,435,668 and 10,987,412, which are hereby incorporated by reference in their entireties.

In some embodiments, the aAPCs comprise poly(lactic acid)-polyethylene glycol (PLA-PEG) or poly(lactic acid-co-glycolic acid)-polyethylene glycol (PLGA-PEG) co-polymers. PLA-PEG and PLGA-PEG nanoparticles can be prepared by nanoprecipitation using known processes. In these embodiments, the aAPCs have advantages in stability and ligand density, among other things. The aAPC is generally suitable for parenteral administration (including subcutaneous administration in some embodiments), and may comprise PLA-PEG or PLGA-PEG copolymers and one or more polypeptide ligands conjugated to PEG (e.g., conjugated to the PEG terminus) through a thioether bond or other conjugation chemistry. The polypeptide ligands comprise HLA ligands (Human Leukocyte Antigen ligands) presenting a peptide antigen (i.e., a target antigen), and one or more signal 2 ligands for T cell activation. In various embodiments, about 40% or less by weight of the copolymers have a functional group for polypeptide ligand coupling. In various embodiments, about 30% or less, or about 25% of less, or about 20% or less by weight of the copolymers have a functional group for polypeptide ligand coupling. In some embodiments, from about 15% to about 35% by weight of the copolymers have a functional group for polypeptide ligand coupling, such as maleimide. In various embodiments, PEG-maleimide (by weight of the composition) is less than about 10%, or less than about 7%, such as about 5%.

In various embodiments, the ratio of PEG-maleimide groups to end-capped PEG (inert) limits the density of the polypeptide ligands conjugated through sulfhydryl groups. In various embodiments, the aAPC has about 10 to about 500 polypeptide ligands per particle (on average). In some embodiments, the aAPC has about 50 to about 400 polypeptide ligands per particle (on average). In some embodiments, the aAPC has about 100 to about 300 polypeptide ligands per particle (on average).

In various embodiments, the PLA or PLGA portion of the copolymers have molecular weights of from about 15 kDa to about 50 kDa. In some embodiments, the PLA or PLGA portion of the copolymers have molecular weights in the range of about 15 kDa to about 35 kDa, or from about 15 kDa to about 25 kDa. In an exemplary embodiment, the PLA or PLGA portion of the copolymers have a molecular weight of about 20 kDa. In various embodiments, the PEG portions of the copolymers have molecular weights in the range of about 2 kDa to about 10 kDa, or in the range of about 2 kDa to about 7 kDa. In an exemplary embodiment, the PEG portion of the copolymers have molecular weights in the range of about 2 kDa and about 5 kDa. In some embodiments, the PEG portions having a functional group for polypeptide ligand coupling have molecular weights of about 5 kDa, and the PEG portions without functional groups for ligand coupling have molecular weights of about 3 kDa. Thus, in exemplary embodiments, the PLA or PLGA portions of the copolymers have molecular weights of about 20 kDa, the PEG-maleimide portions of the co-polymers have molecular weights of about 5 kDa, and the mPEG (end capped PEG) portions of the co-polymers have molecular weights of about 3 kDa. It is understood that any conjugation chemistry may be employed in the various aspects and embodiments, including click chemistry.

In these or other embodiments, the nanoparticles comprise a PEG-conjugated lipid. Exemplary PEG lipids are selected from one or more of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol. A PEG lipid may be selected from PEG-c-DOMG, PEG-DMG, PEG-DLPE, PEG-DMPE, PEG-DPPC, PEG-Cholesterol, PEG tocopherol, or a PEG-DSPE lipid.

In some embodiments, the nanoparticles further comprise a cationic or ionizable lipid, optionally with one or more of a neutral lipid or phospholipid, and a structural lipid. Exemplary structural lipids can be selected from one or more of cholesterol, fecosterol, sitosterol, ergosterol, campesterol, stigmasterol, brassicasterol, tomatidine, ursolic acid, and tocopherols (e.g., alpha tocopherol). In some embodiments, the structural lipid is cholesterol. In some embodiments, the LNP comprises one or more phospholipids. Exemplary phospholipids are selected from the group consisting of cardiolipins, sterol modified lipids (modified with a cholesterol moiety attached at the sn-2 carbon of the glycerol backbone), mixed-acyl glycerophospholipids, and symmetrical acyl glycerophospholipids. Head groups for acyl glycerophospholipids include, for example, phosphatidic acid, lysophosphatidic acid, phosphatidylcholine, phosphatidylethanolamine, phosphatidylglycerol, phosphoinositides, and phosphatidylserine. Exemplary phospholipids are selected from 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanol amine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the nanoparticle is a hybrid particle based on PLA/PLA-PEG or PLGA/PLGA-PEG as described, and engineered to be more fusogenic, such as by addition of an ionizable or cationic lipid (as described above), as well as optionally one or more other components of lipid nanoparticles, such as a neutral lipid or phospholipid, and a structural lipid. In these embodiments, a hybrid polymeric/lipid nanoparticle supports both T cell activation and fusogenic properties. That is, the polymeric and/or lipid components of the nanoparticle can be selected to be fusogenic, and therefore allow for the ligands to activate the cognate T cell, followed by fusion of the nanoparticle with the cell membrane to deliver a nucleic acid (such as an mRNA) encoding the BiTE to the cell. This bimodal design allows for both modalities to be delivered in a single composition. A biphasic response would be expected, where T cell activation and expansion occurs first, followed by BiTE expression and secretion into the circulation about 12 hours later and continuing for several days.

mRNA therapy employing stabilized or modified mRNAs as is known in the art (including with modified nucleotides such as pseudouridine or N1 methyl-pseudouridine), and lipid nanoparticles for delivery thereof, are described in U.S. Pat. Nos. 9,738,593; 9,867,888, 10,221,127; 10,166,298; 10,266,485; and 10,442,756, which are each hereby incorporated by reference in their entireties. For example, the nanoparticles may comprise: an ionizable lipid; one or more selected from myristoylphosphatidylcholine (DMPC), dipalmitoylphosphatidylcholine (DPPC), and distearoylphosphatidylcholine (DSPC); cholesterol; and a PEG-lipid. In various embodiments, polynucleotides encoding one or more BiTEs are encapsulated by the aAPC or are conjugated to the surface of the aAPC, and expressed following internalization of the aAPC by target T cells. Means for surface conjugation and modifications to stabilize the RNA for surface conjugation are known, and include those described in Rouge et al., Spherical Nucleic Acids as a Divergent Platform for Synthesizing RNA-Nanoparticle Conjugates through Enzymatic Ligation. ACS Nano 2014, 8, 9, 8837-8843.

In some embodiments, antigen presenting complexes (e.g., such as aAPCs) are administered separately from a particle encapsulating or conjugating one or more polynucleotides encoding one or more BiTEs. Such particles include LNPs as described above. Such particles can be administered via any route, including subcutaneously, intravenously, or intramuscularly, to release BiTEs transiently in the circulation for one or several days. When the compositions are administered simultaneously, the biphasic response will provide for BiTE expression and secretion after initial T cell activation and expansion has occurred. Constructs for expressing antibody heavy and light chains simultaneously in cells (e.g., muscle cells) are known in the art.

In various embodiments, the aAPC has a diameter of from about 50 nm to about 150 or a diameter of from about 50 nm to about 130 nm. In some embodiments, the aAPC has a diameter of from about 50 nm to about 120 nm. In some embodiments, the aAPC has a diameter of from about 50 nm to about 100 nm or from about 50 nm to about 75 nm. In some embodiments, the aAPC population has a size distribution with polydispersity index (PDI) of less than 0.2. In various embodiments, the aAPC has a surface charge of from about 0 to −15 mV, or from about 0 to about-10 m V. For example, the aAPC may have a surface charge of from about-2.5 m V to about-10 mV. The aAPC size and surface charge allows for desired circulating and biodistribution properties, and in some embodiments provides advantages in particle stability.

The HLA ligands are generally HLA Class I molecular complexes. In some embodiments, the HLA molecular complexes are monomeric or dimeric, and may contain additional heterologous sequences, such as immunoglobulin sequences. HLA-fusions (e.g., HLA-Immunoglobulin fusions) in some embodiments provide additional advantages in stability, TCR binding affinity, and/or potency for T cell activation. In some embodiments, the HLA class I ligand comprises at least two fusion proteins. A first fusion protein comprises a first HLA class I a chain and a first immunoglobulin heavy chain, and a second fusion protein comprises a second HLA class I a chain and a second immunoglobulin heavy chain. The first and second immunoglobulin heavy chains associate to form the HLA class I molecular complex (e.g., associate through disulfide bonds). The HLA class I molecular complex comprises a first HLA class I peptide binding cleft and a second HLA class I peptide binding cleft.

In various embodiments, the immunoglobulin sequence of the HLA polypeptide ligand (i.e., HLA-Ig) is a partial heavy chain sequence comprising the hinge region to support dimerization. In some embodiments, the HLA-Ig fusion construct contains no variable region sequences. For example, the HLA extracellular domain sequences (i.e., HLA class I alpha chain extracellular domain) can be fused to an Ig constant region sequence above the hinge region to provide a dimeric HLA. For example, an HLA or antigen presenting portion thereof may be conjugated to a CH1 portion of each IgG heavy chain. All IgG molecules consist of two identical heavy chains (constant and variable regions) joined together by disulfide bonds in the hinge region (upper and lower). For example, in some embodiments, an HLA molecule or antigen presenting complex is fused to the CH1 (N-terminal end of the Ig heavy chain above the hinge region), thereby creating a dimeric fusion protein that is smaller than fusion to the ends of full antibody heavy chains, due to lack of any VH and VL light chain sequences. Thus, such constructs would further include CH2 and CH3 domains. Such a construct provides manufacturing advantages, as well as exhibits less potential for immunogenicity. In some embodiments, these constructs also display sufficient binding cooperativity for efficient T cell activation or inhibition.

In various embodiments, the immunoglobulin heavy chain sequences fused to the HLA Class I can be any isotype, and in some embodiments are IgG. In some embodiments, the isotype is selected from IgG1, IgG3, IgG2B, IgG2a, and IgG4. In some embodiments, the immunoglobulin sequences are IgG4 Fc sequences.

In some embodiments, the recombinant HLA ligand comprises a linker between the HLA amino acid sequence, and the immunoglobulin sequences (e.g., IgG4 Fc domain). In some embodiments, the linker is a flexible linker, such as a linker that is predominately glycine and serine amino acid residues. Linkers can be selected from flexible and rigid peptide linkers. Flexible linkers are predominately or entirely composed of small and/or polar residues such as Gly, Ser, and Thr. An exemplary flexible linker comprises (GlyₓSer)ₙ linkers, where x is from 1 to 10 (e.g., from 2 to 6), and n is from 1 to about 10, and in some embodiments, is from 2 to about 6. In exemplary embodiments, x is from 2 to 4, and n is from 2 to 4. Due to their flexibility, these linkers are substantially unstructured. More rigid linkers include polyproline or poly Pro-Ala motifs and α-helical linkers. Generally, linkers of varying rigidity can be predominately composed of amino acids selected from Gly, Ser, Thr, Ala, and Pro. Exemplary linker sequences contain at least 5 amino acids, and may be in the range of 5 to 30 amino acids or in the range of 5 to 20 amino acids.

In some embodiments, the HLA ligands are selected from HLA-A, HLA-B, HLA-C, or HLA-E ligands. In embodiments, the HLA ligand comprises associated beta 2 microglobulin (β2M) polypeptide. In various embodiments, the HLA ligand (e.g., as presented by an HLA-Ig) corresponds to an allele selected from HLA-A*02:01, HLA-A*01:01, HLA-A*02:05, HLA-A*02:06, HLA-A*02:12, HLA-A*03:01, HLA-A*11:01, HLA-A*24:02, and HLA-B*07:02.

In some embodiments, the aAPCs contain HLA-E ligands as described in PCT/US2022/37279, which is hereby incorporated by reference in its entirety. For example, the HLA-E ligands may be engineered to reduce or eliminate interaction with NKG2A/CD94. HLA-E is a non-classical MHC Class I molecule and is represented by only two principal alleles. Given this low polymorphism, HLA-E ligands may be adaptable to create a nearly universal aAPC platform. However, HLA-E has a dual role in both the innate and adaptive immune systems. The role of HLA-E in the innate immune response is to present peptides of other HLA class I molecules to inhibit Natural Killer (NK) cell-mediated lysis via recognition by NKG2A/CD94. NK cells sense the presence of HLA-E presenting self-peptides, and thereby receive inhibitory signals through the NKG2A/CD94 complex (inhibiting NK-mediated lysis). HLA-E can also bind and present peptide sequences for recognition by T-cells (e.g., CD8+ T cells) (the adaptive immune response). Notably, the HLA-E molecule binds NKG2A/CD94 through a binding surface that overlaps with the binding surface for interacting with the T-cell Receptor (“TCR”).

In various embodiments, the NK cell deactivating function of HLA-E is decoupled from the T cell activating function, by engineering point mutations that affect only the HLA-E binding to NKG2A/CD94, but not the binding of HLA-E to the TCR. These point mutations enable the redirection of HLA-E for modulating HLA-E-restricted T cells, while avoiding HLA-E exhaustion of Natural Killer cells. In accordance with other aspects and embodiments, HLA-E amino acid substitutions are implemented to provide a stable peptide-binding cleft for presentation of bound antigen to HLA-E-restricted T cells.

Peptide antigens are bound to an antigen binding cleft of the antigen presenting complex. Optionally, an antigenic peptide can be covalently bound to a peptide binding cleft. If desired, a peptide tether can be used to link an antigenic peptide to a peptide binding cleft. For example, crystallographic analyses of multiple class I MHC molecules indicate that the amino terminus of β2M is very close, approximately 20.5 Angstroms away, from the carboxyl terminus of an antigenic peptide resident in the MHC peptide binding cleft. Thus, using a relatively short linker sequence, approximately 13 amino acids in length, one can tether a peptide to the amino terminus of β2M. If the sequence is appropriate, that peptide will bind to the MHC binding groove. Peptide antigens for immune therapy of oncological disease, infectious diseases, and autoimmune diseases are described herein.

In some embodiments, the polypeptide ligands of the antigen presenting composition comprise a signal 2 ligand that is a co-stimulatory ligand. Exemplary co-stimulatory ligands include agonists for any one of CD28, 4-1BB, CD27, OX-40, CD30, ICOS, and LIGHT, among others. Antibody agonists can be full monoclonal antibodies, or antigen-binding portions thereof, such as Fab, Fab′, F(ab′) 2 or scFv. Any antigen-binding platform can be employed, including single chain antibodies, nanobodies, and adnectins.

In various embodiments, the signal 1 and signal 2 ligands can be combined in homodimeric or heterodimeric constructs (e.g., homodimeric or heterodimeric Ig fusion constructs). For example, the HLA ligand can comprise fusion of HLA extracellular domains to an immunoglobulin Fc region, such as IgG4 Fc region, which can be dimerized (e.g., through disulfide bonds) with a signal 2-Immunoglobulin (Ig) fusion (i.e., a heterodimeric Ig fusion construct). In still other embodiments, the HLA ligand comprises a fusion to a signal 2 ligand. For example, an HLA extracellular domain can be fused at its C-terminus to an immunoglobulin Fc region (e.g., IgG4 Fc as already described), and fused at its N-terminus to a signal 2 ligand, to prepare homodimeric ligands with both signals dimerized. In some embodiments, the signal 2 ligand comprises a single chain antibody (e.g., scFv) or agonistic fraction of a natural ligand.

In some embodiments, the co-stimulatory ligand is an agonistic antibody against CD28, which is optionally a humanized or human monoclonal antibody or a scFv based thereon. For example, the anti-CD28 antibody may be an IgG isotype (e.g., IgG4), and may be as described in U.S. Pat. No. 10,632,193, which is hereby incorporated by reference in its entirety. In some embodiments, one, two, three, or more complementarity determining regions (CDRs) are based on mouse 9.3 mAb (Tan et al. J. Exp. Med. 1993 177:165). In some embodiments, the antibody has the full set of heavy chain and/or full set of light chain CDRs of 9.3 mAb. For example, in some embodiments the heavy chain variable region contains one, two or three of the following CDRs, which optionally may each be modified by one, two, or three amino acid substitutions: CDR1 (DYGVH, SEQ ID NO: 1), CDR2 (VIWAGGGTNYNSALMS, SEQ ID NO: 2), and CDR3 (DKGYSYYYSMDY, SEQ ID NO: 3). In some embodiments, the light chain contains one, two, or three of the following CDRs, which each may be modified by one, two, or three amino acid substitutions: CDR1 (RASESVEYYVTSLMQ, SEQ ID NO: 4), CDR2 (AASNVES, SEQ ID NO: 5), and CDR3 (QQSRKVPYT, SEQ ID NO: 6). In some embodiments, the anti-CD28 antibody (or portion thereof) binds to the same or overlapping epitope as 9.3 mAb, or binds the same or overlapping epitope as an antibody having CDR1, CDR2, and CDR3 of 9.3 mAb. Antibodies with the same or overlapping epitope can be selected by any suitable technique, including competitive immunoassays, using, for example, Surface Plasmon Resonance (Biacore). Alternative CDR sequences, variable regions, or CD28-binding ligands may be employed in various embodiments. Alternative ligands, CD28 epitopes, and anti-CD28 antibodies are described in U.S. Pat. Nos. 7,612,170, 6,987,171, and 6,887,466, for example, and these disclosures are hereby incorporated by reference in their entireties.

In some embodiments, the aAPC further comprises one or more cytokines that support T cell activation and/or expansion. The one or more cytokines or functional portion thereof may be conjugated to the aAPC as a polypeptide ligand. Alternatively, the cytokine or functional portion thereof may be fused to a signal 1 or signal 2 polypeptide ligand (which can optionally be presented in homodimeric or heterodimeric Ig fusion constructs as described herein). In some embodiments, the cytokine (or mRNA encoding the cytokine) is encapsulated by the copolymers, and will release cytokine locally in targeted environments (e.g., in lymphoid organs, tumor, or target tissue or organ). Examples of cytokines that may be used include IL-1B, IL-2, IL-4, IL-7, IL-12, IL-15, and gamma interferon. For example, IL-2 can be employed with a co-stimulatory signal 2 ligand.

In some embodiments, the ligands further comprise one or more homing ligands for lymphoid organs. For example, an exemplary homing ligand is CD62L. In some embodiments, ligands (which may or may not be polypeptide ligands) are included to target the aAPCs to a tissue or organ or interest, such as the pancreas, intestine, lungs, liver, muscle, skin, etc. Suitable peptide ligands or other ligands can be selected based on information in the art.

In other embodiments, the antigen presenting composition comprises a composition or complex described in U.S. Pat. Nos. 10,927,158, 10,927,161, 11,104,712, PCT/US2017/020480, PCT/US2017/020276, PCT/US2017/033187, PCT/US2018/012830, PCT/US2018/049760, PCT/US2019/012688, and PCT/US2018/022492, which are hereby incorporated by reference in their entireties. For example, the antigen presenting composition may comprise T-cell modulatory multimeric polypeptides to functions as a surrogate APC. The multimeric polypeptide does so by engaging a TCR present on the surface of a T cell with an epitope-presenting peptide complexed with an MHC present in the multimeric polypeptide. The multimeric polypeptide also comprises at least one immunomodulatory protein that engages a counterpart costimulatory protein on the T cell (e.g., CD38). The multimeric protein may also comprise a variant of a naturally occurring immunomodulatory protein (e.g., naturally occurring IL-2), which variant exhibits a reduced affinity for its receptor on the T cell (e.g., IL-2R) as compared to the affinity of the naturally occurring immunomodulatory protein for the receptor. These signals (often referred to as Signal 1, Signal 2, and Signal 3) can be combined in various fusion protein formats for administration.

In certain embodiments, the antigen presenting composition may employ enucleated cells as described in US 2019/0290686, which is hereby incorporated by reference in its entirety.

In the various aspects described above, the one or more peptide antigens (i.e., target antigens) can be tumor or cancer associated antigens, such as tumor-derived antigens, tumor-specific antigens, and neoantigens. T cells specific for tumor associated antigens are often very rare, and in many cases undetectable, in the peripheral blood of healthy individuals. Further, the cells are often of a naive phenotype. See, Quintarelli et al., Cytotoxic T lymphocytes directed to the preferentially expressed antigens of melanoma (PRAME) target chronic myeloid leukemia. Blood 2008; 112:1876-1885. This is often a distinction observed between viral-specific and tumor antigen specific T cells.

“Tumor-associated antigens” or “cancer specific antigens” include unique tumor or cancer antigens expressed exclusively by the tumor or malignant cells from which they are derived, shared tumor antigens expressed in many tumors but not in normal adult tissues (oncofetal antigens), and tissue-specific antigens expressed also by the normal tissue from which the tumor arose. Tumor associated antigens can be, for example, embryonic antigens, antigens with abnormal post-translational modifications, differentiation antigens, products of mutated oncogenes or tumor suppressors, fusion proteins, or oncoviral proteins.

A variety of tumor-associated antigens are known in the art. Oncofetal and embryonic antigens include carcinoembryonic antigen and alpha-fetoprotein (usually only highly expressed in developing embryos but frequently highly expressed by tumors of the liver and colon, respectively), MAGE-1 and MAGE-3 (expressed in melanoma, breast cancer, and glioma), placental alkaline phosphatase sialyl-Lewis X (expressed in adenocarcinoma), CA-125 and CA-19 (expressed in gastrointestinal, hepatic, and gynecological tumors), TAG-72 (expressed in colorectal tumors), epithelial glycoprotein 2 (expressed in many carcinomas), pancreatic oncofetal antigen, 5T4 (expressed in gastriccarcinoma), alphafetoprotein receptor (expressed in multiple tumor types, particularly mammary tumors), and M2A (expressed in germ cell neoplasia).

Mutated oncogene or tumor-suppressor gene products include Ras and p53, both of which are expressed in many tumor types, Her-2/neu (expressed in breast and gynecological cancers), EGF-R, estrogen receptor, progesterone receptor, retinoblastoma gene product, myc (associated with lung cancer). Fusion proteins include BCR-ABL, which is expressed in chromic myeloid leukemia. Oncoviral proteins include HPV type 16, E6, and E7, which are found in cervical carcinoma.

Tissue-specific antigens include melanotransferrin and MUC1 (expressed in pancreatic and breast cancers); CD10 (previously known as common acute lymphoblastic leukemia antigen, or CALLA) or surface immunoglobulin (expressed in B cell leukemias and lymphomas); the a chain of the IL-2 receptor, T cell receptor, CD45R, CD4+/CD8+ (expressed in T cell leukemias and lymphomas); prostate specific antigen and prostatic acid-phosphatase (expressed in prostate carcinoma); GP100, MelanA/Mart-1, tyrosinase, gp75/brown, BAGE, and S-100 (expressed in melanoma); cytokeratins (expressed in various carcinomas); and CD19, CD20, and CD37 (expressed in lymphoma).

Tumor-associated antigens also include altered glycolipid and glycoprotein antigens, such as neuraminic acid-containing glycosphingolipids (e.g., GM2 and GD2, expressed in melanomas and some brain tumors); blood group antigens, particularly T and sialylated Tn antigens, which can be aberrantly expressed in carcinomas; and mucins, such as CA-125 and CA-19-9 (expressed on ovarian carcinomas) or the underglycosylated MUC-1 (expressed on breast and pancreatic carcinomas).

Tumor-associated antigens are further disclosed in U.S. Pat. No. 11,007,222, which is hereby incorporated by reference.

In some embodiments, the target peptide antigens include at least one that is associated with or derived from a pathogen, such as a viral, bacterial, fungal, or parasitic pathogen. For example, at least one peptide antigen may be associated with HIV (human immunodeficiency virus), hepatitis (e.g., A, B, C, or D) cytomegalovirus (CMV), Epstein-Barr virus (EBV), HPV, influenza, herpes virus (e.g., HSV 1 or 2, or varicella zoster), and Adenovirus. CMV, for example, is the most common viral pathogen found in organ transplant patients and is a major cause of morbidity and mortality in patients undergoing bone marrow or peripheral blood stem cell transplants.

In various embodiments, the subject has cancer or an infectious disease. Infectious diseases that can be treated include those caused by bacteria, viruses, prions, fungi, parasites, helminths, etc. Such diseases include human papilloma virus (HPV) (and related cancers), AIDS, hepatitis, EBV infection or associated disease (e.g., multiple sclerosis or PTLD), CMV infection, and post-transplant lymphoproliferative disorder (PTLD). CMV, for example, is the most common viral pathogen found in organ transplant patients and is a major cause of morbidity and mortality in patients undergoing bone marrow or peripheral blood stem cell transplants. This is due to the immunocompromised status of these patients, which permits reactivation of latent virus in seropositive patients or opportunistic infection in seronegative individuals.

PTLD occurs in a significant fraction of transplant patients and results from Epstein-Barr virus (EBV) infection. EBV infection is believed to be present in approximately 90% of the adult population in the United States. Active viral replication and infection is kept in check by the immune system, but, as in cases of CMV, individuals immunocompromised by transplantation therapies lose the controlling T cell populations, which permits viral reactivation. This represents a serious impediment to transplant protocols. EBV may also be involved in tumor promotion in a variety of hematological and non-hematological cancers. There is also a strong association between EBV and nasopharyngeal carcinomas.

Cancers that can be treated according to this disclosure include melanoma, carcinomas, e.g., colon, head and neck cancer, duodenal, prostate, breast, lung, ovarian, ductal, hepatic, pancreatic, renal, endometrial, stomach, dysplastic oral mucosa, polyposis, invasive oral cancer, non-small cell lung carcinoma, transitional and squamous cell urinary carcinoma etc.; neurological malignancies, e.g., neuroblastoma, gliomas, etc.; hematological malignancies, e.g., chronic myelogenous leukemia, childhood acute leukemia, non-Hodgkin's lymphomas, chronic lymphocytic leukemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like. See, e.g., Mackensen et al, Int. J. Cancer 86, 385-92, 2000; Jonuleit et al., Int. J. Cancer 93, 243-51, 2001; Lan et al., J. Immunotherapy 24, 66-78, 2001; Meidenbauer et al, J. Immunol. 170 (4), 2161-69, 2003. In some embodiments, the subject has a solid tumor, which can be Stage I, Stage II, Stage III, or Stage IV cancer. In some embodiments, the cancer is metastatic and/or recurrent, and/or is nonresectable. In some embodiments, the patient is refractory or only partially responsive to chemotherapy and/or immune checkpoint inhibitor therapy.

In some embodiments, the therapy is provided together with one or more immune checkpoint inhibitors, such as Nivolumab, Pembrolizumab, and Ipilimumab. In some embodiments, the additional therapy is anti-CTLA4 or anti-PD1, or anti-PD-L1. In some embodiments, the patient is resistant or shows only a partial or transient response to checkpoint inhibitor therapy, and the methods and compositions described herein enhance tumor regression in these patients. In still other embodiments, for cancers that are typically resistant to immune checkpoint inhibitor therapy, the methods and compositions described herein expand the successful use of checkpoint inhibitors to such cancers.

According to the aspects of this disclosure, the BiTE comprises at least a first domain that binds a T cell surface antigen, and a second domain that binds a target cell antigen. In some embodiments, the BiTE is a bispecific antibody. In some embodiments, the BiTE comprises one or more single chain antibodies (scFv) that are dimerized or multimerized. In some embodiments, the first domain and/or the second domain can be natural immune ligands or portions thereof sufficient for binding to the target.

In exemplary embodiments, the first domain binds CD3, and may be an agonist or partial agonist for CD3 in some embodiments. In still other embodiments, the first domain is an HLA-target peptide complex. For example, the HLA-target peptide complexes may present one or more peptide antigens described herein, including but not limited to peptide antigens of WT1, PRAME, Survivin, CyclinA1, XBP1, CD138, CS1, NY-ESO1, SOX2, EBV, CMV, RHAMM, PR3, MART-1/MELAN-A, and gp 100. Where T cells are generated in vivo by administering antigen presenting complexes (e.g., aAPCs), the HLA-target peptide complex employed as the first domain of the BiTE may be the same or substantially the same as that used for the antigen presenting complex. In still other embodiments, the first domain binds CD28 (e.g., anti-CD28 antibody), and is optionally agonistic or non-agonistic for CD28. In some embodiments, the first domain binds CD137 (4-1BB), and in various embodiments is agonistic or non-agonistic. In some embodiments, such stimulatory first domains (e.g., 4-1BBL or agonistic antibody mimicking the same) can be combined with an immune checkpoint blockade as a second domain (as further described below), to provide an “immunoswitch” effect (e.g., convert an immunosuppressive signal on the target cell to an immune stimulatory signal). In still other embodiments, the first domain binds a T cell target selected from PD-1, OX40, CTLA4, TIM3, CD47, CD137, TIGIT, and LAG3. In exemplary embodiments, the first domain binds PD-1 or CTLA4, and blocks binding of PD-L1 to PD-1 or blocks binding of CD80 or CD86 to CTLA-4.

For example, in some embodiments, the subject has a cancer. The cancer may be a hematological malignancy, such as a cancer selected from acute myeloid leukemia, chronic myelogenous leukemia, childhood acute leukemia, non-Hodgkin's lymphomas, acute lymphocytic leukemia, chronic lymphocytic leukemia, myelodysplastic syndrome, malignant cutaneous T-cells, mycosis fungoids, non-MF cutaneous T-cell lymphoma, lymphomatoid papulosis, and T-cell rich cutaneous lymphoid hyperplasia. In such embodiments, the second domain binds a target expressed on the surface of cancer cells. For example, the second domain may bind CD19. In some embodiments, the second domain binds a tumor target selected from BCMA, CD20, GPRC5D, FcRH5, CD38, gpA33, CD33, CD123, CD133, CLE12A, Flt3, and CD79b. In exemplary embodiments, the first domain binds CD3, and the second domain binds CD19, BCMA, CD123, CD133, or Flt3.

In some embodiments, the second domain mimics a T cell receptor, and is specific for an HLA class I peptide antigen complex on the target cell. Peptide antigens associated with various cancers and infectious diseases (e.g., viruses) are well known, and various examples are described herein. Antibodies mimicking TCR can be prepared as described in Duan Z. and Ho M., T cell receptor mimic antibodies for cancer immunotherapy, Mol. Cancer Ther. 2021; 20 (9): 1533-1541. For example, such TCR mimicking antibodies such as ScFv antibodies can be isolated/prepared by phage display using known methods.

In embodiments, the cancer is a solid tumor, such as a cancer selected from a carcinoma or sarcoma. In these embodiments, the second domain binds a target expressed on the surface of cancer cells. Exemplary targets are selected from EpCam (epithelial such as colorectal and liver), PSMA (prostate), GD2 (melanoma neuroblastoma), SSTR2 (pancreas, kidney, liver, GI), MUC16 (epithelial), MUC17 (epithelial, GI), 5T4 (epithelial), B7-H4 (epithelial, breast), MSLN (pancreas, ovarian, mesothelioma), CD47 (bladder), EGFR, CLDN (gastric), B7-H3 (breast), survivin, CDH19 (melanoma, GBM), HER2 (epithelial, breast), HER3 (epithelial, breast), GUCY2c (colorectal), DLL3 (SCLC, neuroendocrine), STEAP1 (prostate), CLDN6, ROR1 (ovarian), CD38, and SIGLEC6 (colorectal). In some embodiments, such targets on the tumor are targeted via a TCR mimicking antibody as described above, in which a relevant peptide antigen is complexed with HLA on the cell surface.

In still other embodiments, the target for the second domain is PD-L1, which can be targeted via antibody binding (e.g., anti-PD-L1 antibody) or natural ligand or PD-L1-binding portion thereof (e.g., PD-1 or portion thereof). In certain embodiments, anti-PD-L1 (or PD-L1 binding protein) is coupled with a co-stimulatory domain, such as 4-1BBL or agonistic portion thereof.

BiTEs in various embodiments may be bispecific antibodies, ScFvs linked in tandem (optionally with a half-life extending domain, such as IgG Fc domain), natural ligands or portions thereof dimerized by fusion to immunoglobulin chains comprising an Fc domain (e.g., comprising an antibody hinge region) or dimerized through a peptide linker (e.g., Gly Ser linker), or may employ dual affinity retargeting (DART) configuration. See Moore PA, Application of dual affinity retargeting molecules to achieve optimal redirected T-cell killing of B-cell lymphoma. Blood Vol. 117, Issue 17, 28 Apr. 2011. In DART proteins, each Fv is formed by the association (e.g., linkage) of a VL partner on one chain with a VH partner on the second chain in a VL_{A}−VH_{B}+VL_{B}−VH_{A} configuration. Exemplary linkers include Gly Ser linkers which are well known. Other configurations for combining two binding domains are well known and can be employed.

In some embodiments, the BiTE is selected from blinatumomab (CD3×CD19), catumaxomab (CD3×EpCAM), mosunetuzumab (CD3×CD20), elranatamab (CD3×BCMA), epcoritamab (CD3×CD20), glofitamab (CD3×CD20), talquetamab (CD3×GPRC5D), teclistamab (CD3×BCMA), flotetuzumab (CD3×CD123), odronextamab (CD3×CD20), plamotamab (CD3×CD20), tarlatamab (CD3×DLL3), vibecotamab (CD3×CD123), acapatamab (CD3×PSMA), cevostamab (CD3×FcRH5), emfizatamab (CD3×CD19×CD137×PDL1), linvoseltamab (CD3×BCMA), nivatrotamab (CD3×GD2), tidutamab (CD3×SSTR2), and ubamatamab (CD3×MUC16).

In exemplary embodiments, the BiTE targets CD137 and PD-L1, either as a bispecific antibody, dimerized scFv, or DART.

In still other embodiments, the second domain binds to a viral protein. Exemplary viral proteins include antigens of hepatitis B virus, papillomavirus, Epstein-Barr virus (EBV), cytomegalovirus (CMV), adenovirus, and HIV, among others.

In various embodiments, the subject is administered the one or more BiTEs during or after administering an initial dose of CTL therapy or antigen presenting composition therapy (as already described). In some embodiments, the subject receives no more than four doses of the CTL therapy or antigen presenting composition therapy. In embodiments, the subject receives one, two, three, or four doses of the CTL therapy or antigen presenting composition therapy. In various embodiments, a dose of the BiTE is administered with each dose of CTL therapy or antigen presenting composition therapy (or with one or two days thereof). In some embodiments, the BiTE and the CTL are administered as a single composition or may be administered as separate compositions. In some embodiments, BiTE therapy is continued after CTL therapy or antigen presenting composition therapy is complete. For example, the BiTE may be administered approximately weekly, biweekly, monthly, or quarterly. In certain embodiments, the BiTE is administered from four to twenty four times, such as from four to twelve times, such as from four to eight times (e.g., 4, 6, 8, 10, or 12 administrations). In some embodiments, the subject further receives an immune checkpoint inhibitor therapy, such as an immune checkpoint inhibitor therapy targeting CTLA-4, PD-1, or PD-L1.

In some aspects, the present disclosure provides a composition comprising a cytotoxic T lymphocyte (CTL) therapy (as described herein) and one or more bispecific T cell engagers (BiTE) (as described herein) in an effective amount to potentiate the CTL therapy, and a pharmaceutically-acceptable vehicle. In various embodiments, the composition comprises a dose of from about 10⁶ to about 10¹⁰ CTLs. In various embodiments, the composition comprises a dose of less than about 10⁹ CTLs, or less than about 10⁸ CTLs, or less than about 5×10⁷ CTLs, or less than about 10⁷ CTLs. In some embodiments, the composition comprises a dose of at least about 10⁷ cells or at least about 10⁸ CTLs.

In accordance with aspects and embodiments of this disclosure, target antigens can be selected for treating the desired condition, many of which are known in the art. In some embodiments, the target peptide antigens include one or more associated with or derived from a hematological malignancy, optionally selected from acute myeloid leukemia, chronic myelogenous leukemia, childhood acute leukemia, non-Hodgkin's lymphomas, acute lymphocytic leukemia, chronic lymphocytic leukemia, myelodysplastic syndrome, malignant cutaneous T-cells, mycosis fungoids, non-MF cutaneous T-cell lymphoma, lymphomatoid papulosis, and T-cell rich cutaneous lymphoid hyperplasia.

In some embodiments, one or more peptide antigens are selected from WT1, PRAME, Survivin, CyclinA1, XBP1, CD138, CS1, NY-ESO1, SOX2, EBV, CMV, RHAMM, PR3, MART-1/MELAN-A, and gp100.

In some embodiments, the target antigens associated with multiple myeloma are two or more of (or three, four, five, or six of) peptide antigens disclosed in U.S. Pat. No. 9,096,681, which is hereby incorporated by reference in its entirety. Exemplary peptides comprising antigenic epitopes include XBP1 unspliced (UN) 185-193, XBP1-US184-192, XBP1 spliced (SP) 223-231, XBP1-SP₃₆₇₋₃₇₅, CD138₂₆₅₋₂₇₃, CD138₂₆₀₋₂₆₈, CS1₂₄₀₋₂₄₈, CS1₂₃₉₋₂₄₇, NY-ESO1_{157-165A}, and SOX2₁₁₈₋₁₂₇. In some embodiments, the target antigens comprise NY-ESO-1, WT-1, SOX-2, CD138, and CS1. In some embodiments, the target antigens comprise NY-ESO-1, WT-1, SOX-2, CD138, CS1, and XBP1-US and/or XBP1-SP. In some embodiments, the peptide antigens comprise NY-ESO-1, WT-1, and SOX-2. See Table 2.

In some embodiments, one or more target antigens are associated with acute myelogenous leukemia or myelodysplastic syndrome, and may include one or more of (including 1, 2, 3, 4, or 5 of) Survivin, WT-1, PRAME, RHAMM, PR3, and Cyclin A1 antigens. In some embodiments, the target antigens include at least 1, 2, 3, 4, 5, 6, 7, 8, 9, 10 or all target antigens from Table 1 below.

TABLE 1
Exemplary AML target peptide antigens

| | Peptide | | |
|---|---|---|---|
| Antigen | name/position | Sequence | SEQ ID NO: |
| WT-1 | 126-134 | RMFPNAPYL | SEQ ID NO: 7 |
| | 235-243 | CMTWNQMNL | SEQ ID NO: 8 |
| | 37-45 | VLDFAPPGA | SEQ ID NO: 9 |
| | 187-195 | SLGEQQYSV | SEQ ID NO: 10 |
| Prame | P100 | VLDGLDVLL | SEQ ID NO: 11 |
| | P435 | NLTHVLYPV | SEQ ID NO: 12 |
| | P142 | SLYSFPEPEA | SEQ ID NO: 13 |
| | P300 | ALYVDSLFFL | SEQ ID NO: 14 |
| | P425 | SLLQHLIGL | SEQ ID NO: 15 |
| Survivin | ELT 95-104 | ELTLGEFLKL | SEQ ID NO: 16 |
| | LDR 104-113 | LDRERAKNKI | SEQ ID NO: 17 |
| Cyclin A1 | 227-235 | FLDRFLSCM | SEQ ID NO: 18 |
| | 341-351 | SLIAAAAFCLA | SEQ ID NO: 19 |

In some embodiments, one or more target antigens may include one or more of XBP1-US, XBP1-SP, CD138, CS1, NY-ESO1, SOX2, EBV, Influenza, CMV, RHAMM, PR3, Mart-1/Melan A, gp100, CMVpp65, and Influenza Matrix Protein M1 antigens. In some embodiments, the target antigens include at least 1, 2, 3, 4, 5, 6, 7, 8, 9, or 10 target antigens from Table 2 below, which are useful for targeting multiple myeloma, melanoma, or various viral or infectious diseases.

TABLE 2
Target peptide antigens

| | Peptide | | | |
|---|---|---|---|---|
| Antigen | name/position | Sequence | SEQ ID NO: | Restriction |
| XBP1-US | 184-192 | YISPWILAV | SEQ ID NO: 20 | - |
| XBP1-SP | 367-375 | YLFPQLISV | SEQ ID NO: 21 | - |
| CD138 | 260-268 | GLVGLIFAV | SEQ ID NO: 22 | - |
| CS1 | 239-247 | SLFVLGLFL | SEQ ID NO: 23 | - |
| NY-ESO1 | 157-165A | SLLMWITQA | SEQ ID NO: 24 | - |
| SOX2 | 118-127 | ALSPASSRSV | SEQ ID NO: 25 | - |
| LAMP2 | — | CLGGLLTMV | SEQ ID NO: 26 | A2 |
| LAMP2 | — | FLYALALLL | SEQ ID NO: 27 | A2 |
| BMLF1 | — | GLCTLVAML | SEQ ID NO: 28 | A2 |
| BRLF1 | — | YVLDHLIVV | SEQ ID NO: 29 | A2 |
| EBNA3 | — | LLDFVRFMGV | SEQ ID NO: 30 | A2 |
| LMP1 | — | YLQQNWWTL | SEQ ID NO: 31 | A2 |
| LMP2 | — | IYVLVMLVL | SEQ ID NO: 32 | A24 |
| BRLF1 | — | TYPVLEEMF | SEQ ID NO: 33 | A24 |
| BMLF1 | — | DYNFVKQLF | SEQ ID NO: 34 | A24 |
| EBNA3A | — | RYSIFFDYM | SEQ ID NO: 35 | A24 |
| EBNA3B | — | TYSAGIVQI | SEQ ID NO: 36 | A24 |
| EBNA-3A | — | RPPIFIRRL | SEQ ID NO: 37 | B7 |
| EBNA-3C | — | QPRAPIRPI | SEQ ID NO: 38 | B7 |
| BMRF1 | — | RPQGGSRPEFVKL | SEQ ID NO: 39 | B7 |
| M1 | — | GILGFVFTL | SEQ ID NO: 40 | A2 |
| PB1 | — | QPEWFRNVL | SEQ ID NO: 41 | B7 |
| NP | — | SPIVPSFDM | SEQ ID NO: 42 | B7 |
| pp65 | 341-349 | QYDPVAALF | SEQ ID NO: 43 | A24 |
| pp65 | 113-121 | VYALPLKML | SEQ ID NO: 44 | A24 |
| IE-1 | 248-256 | AYAQKIFKI | SEQ ID NO: 45 | A24 |
| pp65 | 417-426 | TPRVTGGGAM | SEQ ID NO: 46 | B7 |
| pp65 | 265-275 | RPHERNGFTVL | SEQ ID NO: 47 | B7 |
| RHAMM | R3 | ILSLELMKL | SEQ ID NO: 48 | — |
| RHAMM | R5 | SLEENIVIL | SEQ ID NO: 49 | — |
| RHAMM | R1 | KLLEYIEEI | SEQ ID NO: 50 | — |
| RHAMM | R2 | KLQEELNKV | SEQ ID NO: 51 | — |
| RHAMM | R8 | KLKGKEAEL | SEQ ID NO: 52 | — |
| PR3 | PR-1₁₆₉₋₁₇₇ | VLQELNVTV | SEQ ID NO: 53 | — |
| Mart-1/ | Mart-1 A27L | ELAGIGILTV | SEQ ID NO: 54 | — |
| Melan A | | | | |
| gp100 | G209-2M, | IMDQVPFSV | SEQ ID NO: 55 | — |
| | gp100 (209-217) | | | |
| NY-ESO 1 | 157-165 | SLLMWITQC | SEQ ID NO: 56 | — |
| NY-ESO 1 | 165A | SLLMWITQA | SEQ ID NO: 57 | — |
| CMVpp65 | pp65 | NLVPMVATV | SEQ ID NO: 58 | — |
| XBP1-UN | 185-193 | ISPWILAVL | SEQ ID NO: 59 | A24 |
| XBP1-SP | 223-231 | VYPEGSSL | SEQ ID NO: 60 | A24 |
| CD138 | 265-273 | IFAVCLVGF | SEQ ID NO: 61 | A24 |
| CS1 | 240-248 | LFVLGLFLW | SEQ ID NO: 62 | A24 |

In some embodiments, one or more target peptide antigens are neoantigens. In some embodiments, between three and ten neoantigens are identified through genetic analysis of the patient's malignancy (e.g., by nucleic acid sequencing of malignant cells), followed by predictive bioinformatics. In some embodiments, the antigens are natural, non-mutated, cancer antigens, of which many are known.

In some embodiments, the second domain of the BiTE binds a target expressed on the surface of cancer cells. Various targets for the second domain are summarized in Table 3:

TABLE 3
BiTE Targets

| Target | | |
|---|---|---|
| Antigen | Cancer Cell Expression | Indication |
| CD19 | B cells | Lymphoma |
| | | Acute lymphoblastic |
| | | leukemia (ALL) |
| | | Chronic lymphocytic |
| | | leukemia (CLL). |
| EpCAM | Epithelial cells | Colorectal cancer |
| | | Liver cancer |
| Her2 | Epithelial cells | Breast Cancer |
| Her3 | Epithelial cells | Breast, Ovarian, Lung, |
| | | Colorectal, Melanoma, |
| | | Head and Neck, Cervical |
| | | and Prostate cancers |
| CD20 | B cells | Lymphoma |
| BCMA | normal and malignant | Multiple Myeloma (MM) |
| | plasma cells | |
| GPRC5D | Epithelial cells | Multiple Myeloma |
| CD123 | leukemic stem cells (LSCs) | Hematolymphoid |
| | and more differentiated | neoplasms, including acute |
| | leukemic blasts | myeloid leukemia, blastic |
| | | plasmacytoid dendritic cell |
| | | neoplasm, acute |
| | | lymphoblastic leukemia, |
| | | hairy cell leukemia, and |
| | | systemic mastocytosis |
| DLL3 | small cell lung cancer cells | small cell lung cancer, |
| | | neuroendocrine tumors |
| PSMA | Endothelial cells | Prostate cancer |
| FcRH5 | B cells and plasma cells | Myeloma cells |
| PDL1 | Expressed on cancer cells, | Wide range of tumor cells, |
| | stromal cells and immune | including lung cancer, |
| | cells, including infiltrating | breast cancer and melanoma |
| | myeloid and T cells | |
| CD137 | T lymphocytes and natural | Multiple cancers |
| | killer cells | |
| GD2 | various types of malignant | neuroblastomas, |
| | cells | melanomas, |
| | | retinoblastomas, Ewing |
| | | sarcomas, small cell lung |
| | | cancer, gliomas, |
| | | osteosarcomas, and soft |
| | | tissue sarcoma |
| SSTR2 | Epithelial cells | pancreas, kidney, liver, GI, |
| | | breast cancer, |
| | | neuroendocrine neoplasms, |
| | | and small-cell lung |
| | | carcinomas |
| MUC16 | Epithelial cells, bronchial, | Multiple cancers, including |
| | endometrial, ovarian and | ovarian cancer |
| | corneal epithelial cells | |
| 5T4 | Epithelial cells | Multiple cancers |
| B7-H4 | Epithelial cells, T cells, B | several human cancers, |
| | cells, monocytes, and | including breast cancer |
| | dendritic cells | |
| MSLN | mesothelial cells and | pancreatic cancers, ovarian |
| | epithelial cells | cancers, mesotheliomas, and |
| | | other cancers |
| CD38 | bone marrow cells, natural | Multiple cancers |
| | killer cells, monocytes, and | |
| | activated T- and B- | |
| | lymphocytes | |
| CD47 | Expressed in multiple | Multiple cancers, including |
| | cancer cells | bladder cancer |
| gpA33 | epithelial cells | Multiple cancers |
| CD33 | myeloid progenitor cells | Acute myeloid leukemia |
| EGFRvIII | Expressed in multiple | Multiple cancers |
| | cancer cells | |
| CLDN | Expressed in multiple | Multiple cancers, including |
| | cancer cells | gastric cancer |
| B7H3 | tumor and stromal cells | Small cell lung cancer, non- |
| | | small cell lung cancer, |
| | | breast cancer, and other |
| | | cancers |
| CLEC12A | Leukemia cells | Acute myeloid leukemia |
| MUC17 | Epithelial cells, tumor cells, | Multiple cancers, including |
| | including pancreatic tumor | gastric cancer, pancreatic |
| | cells | cancer |
| Survivin | Expressed in multiple | Multiple cancers |
| | cancer cells | |
| Flt3 | Expressed in leukemia cells | leukemia |
| STEAP1 | Expressed in multiple | Multiple cancers, including |
| | cancer cells | prostate cancer and breast |
| | | cancer |
| CLDN6 | Epithelial cells | Multiple cancers, including |
| | | non-small-cell lung cancer |
| | | and ovarian cancer. |
| EGFR | Expressed in multiple | Multiple cancers, including |
| | cancer cells | lung, head and neck, colon, |
| | | pancreas, breast, ovary, |
| | | bladder and kidney, and in |
| | | glioma. |
| PSCA | basal cells | Prostate cancer |
| ROR1 | Expressed in multiple | B-cell chronic lymphocytic |
| | cancer cells | leukemia ( |
| CD79b | lymphoma subtypes | B-cell NHL malignancies |
| PD-1 | Expressed on T cells, B | Wide range of tumor cells, |
| | cells, and monocytes | including melanoma, |
| | | hepatocellular carcinoma |
| | | (HCC), and NSCLC. |
| GUCY2 | Epithelial cells | Colorectal cancer |
| CDH19 | Cancer cells | melanoma, GBM |
| SIGLEC6 | Cancer cells | Colorectal, chronic |
| | | lymphocytic leukemia |

The singular forms “a,” “an,” and “the” include plural referents unless the context clearly dictates otherwise.

As used in the specification and in the claims, the open-ended transitional phrases “comprise(s),” “include(s),” “having,” “contain(s),” and variants thereof require the presence of the named features/steps and permit the presence of other features/steps. These phrases should also be construed as disclosing the closed-ended phrases “consist of” or “consist essentially of” that permit only the named features/steps and unavoidable impurities, and exclude other features/steps.

As used herein, the term “about” means ±10% of a numerical value, unless the context requires otherwise.

The term CDR refers to a complementarity-determining region. CDRs are part of the variable chains in immunoglobulins (antibodies). A set of CDRs constitutes a paratope.

This invention is further illustrated by the following non-limiting examples.

Other aspects and embodiments of the invention will be apparent to the skilled artisan.

### EXAMPLES

### Example 1: Combination of Multiple Myeloma Specific CTLs and BiTE Potentiate Target Killing

The experiments of this example demonstrate that a BiTE significantly increases potency of target killing by target-specific CTLs. In these experiments, a cytotoxicity assay was performed using effector cells (E) mixed with target cells (T).

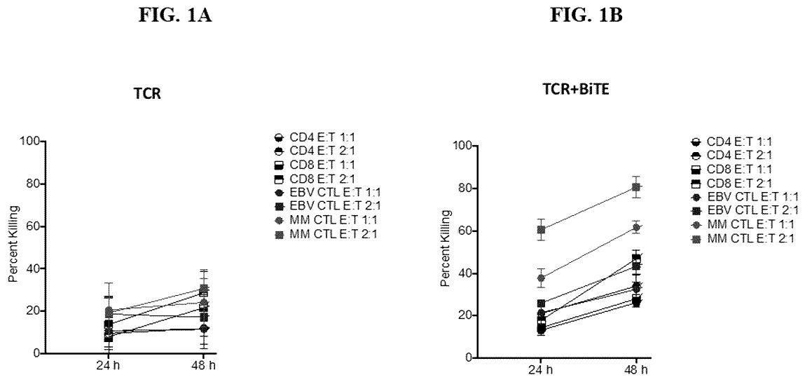

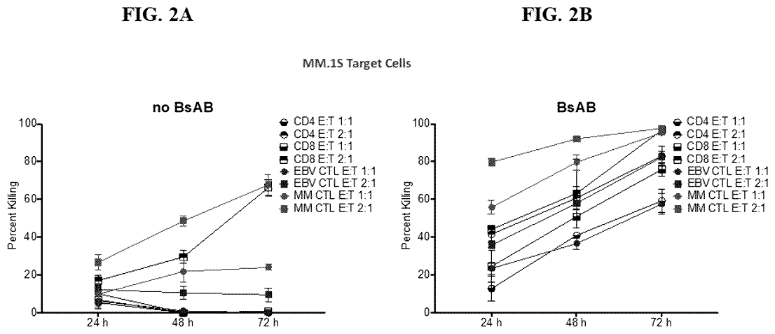

FIG. 1A and FIG. 1B show results of a cytotoxicity assay using various effector cells (E) mixed with target cells (T) (U266 cells, a Multiple Myeloma cell line) at the listed E:T ratios. Effector cell populations were: CD4+ cells isolated from peripheral blood, CD8+ cells isolated from peripheral blood, EBV-specific CTLs, and Multiple Myeloma (MM) specific CTLs. The EBV and MM specific CTLs were prepared by ex vivo expansion with artificial Antigen Presenting Cells (aAPCs) essentially as described in U.S. Pat. Nos. 10,987,412 and 11,007,722, which are hereby incorporated by reference in their entireties. FIG. 1A shows that the effector cells generated minimal killing, although as expected the MM-specific CTLs produced the highest level of killing, reaching about 30% target cell killing at 48 hours. FIG. 2B shows the same experiment with the addition of a CD3×BCMA BITE (0.8 μM). As shown, the MM-specific CTLs with BiTE produced a dramatic killing effect, reaching nearly 80% target killing at 72 hours (2:1 E:T). Other non-specific effector cells also showed some increased killing. FIG. 2A and FIG. 2B show the same effect using MM. 1S target cells.

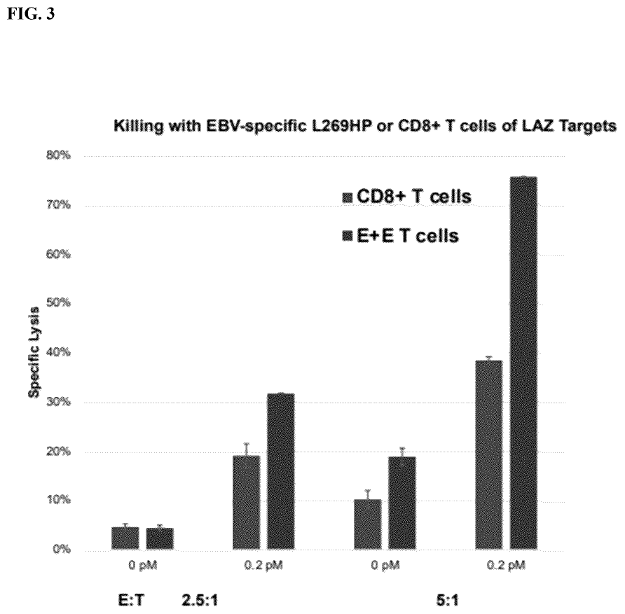

FIG. 3 shows similar results using an EBV-infected cell line (LAZ) as target. EBV-specific CTLs produced ex vivo using the Enrichment+Expansion (E+E) process (see U.S. Pat. Nos. 10,987,412 and 11,007,722) were substantially potentiated by CD3×BCMA BiTE at 0.2 μM. Bulk (e.g., uneducated) CD8+ cells produce low killing. At 5:1 E:T ratio E+E cells (with 0.2 μM BiTE) produced about 75% killing with overnight incubation. Without BiTE, neither cells produced appreciable killing at 2.5:1 E:T ratio.

Collectively, the experiments of this example demonstrate that adoptive cell therapy with BiTE administration could substantially increase both the potency and specificity of cellular cytotoxicity for target cells.

### Example 2: Dose Responses and Cellular Cytotoxicity

The experiments of this example examined various BiTE titrations and dose responses for cellular cytotoxicity of AML target cells.

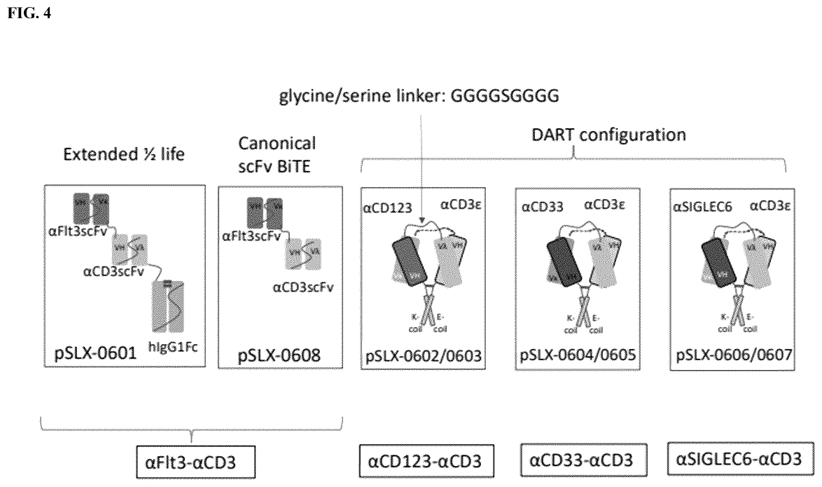

FIG. 4 is an image showing various BiTE designs: αFlt3-αCD3, αCD123-αCD3, αCD33-αCD3, αSIGLEC6-αCD3

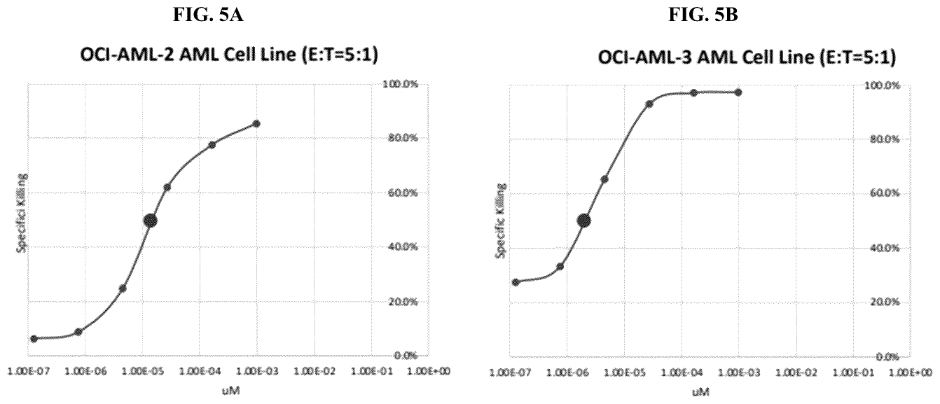

A dose titration of the Flt3 BiTE is shown in FIG. 5A and FIG. 5B against HLA-A2+ AML cell lines OCI-AMI-2 and OCI-AMI-3, respectively. E+E cells specific for AML (1 target) were added at 5:1 E:T.

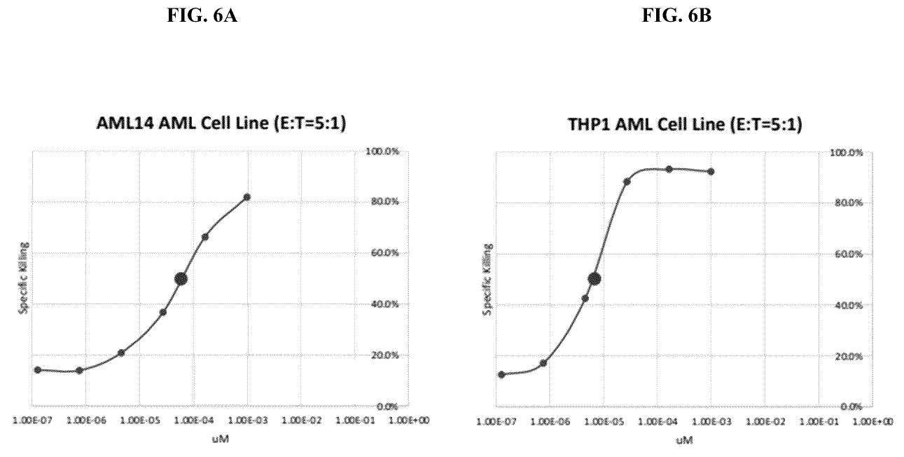

A dose titration of the Flt3 BiTE is shown in FIG. 6A and FIG. 6B against HLA-A2+ AML cell lines AML 14 and THP1, respectively. E+E cells specific for AML (2 targets) were added at 5:1 E:T.

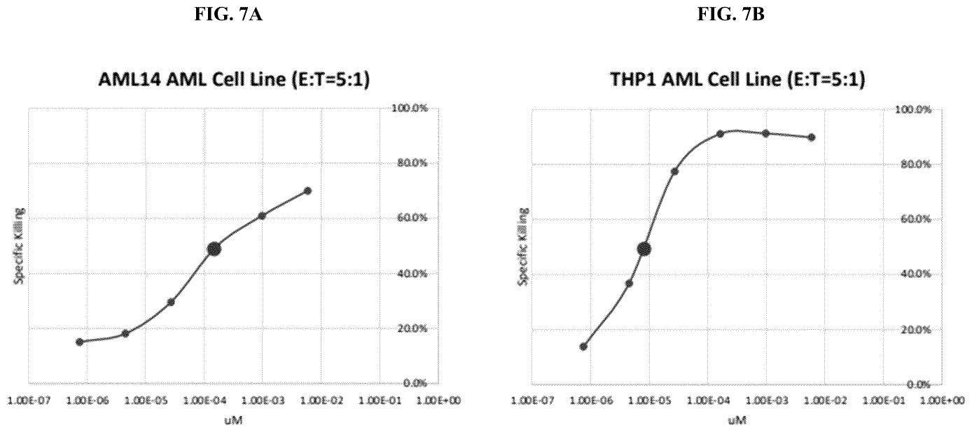

A dose titration of the CD133 BiTE is shown in FIG. 7A and FIG. 7B HLA-A2+ AML cell lines AML 14 and THP1, respectively. E+E cells specific for AML (2 targets) were added at 5:1 E:T.

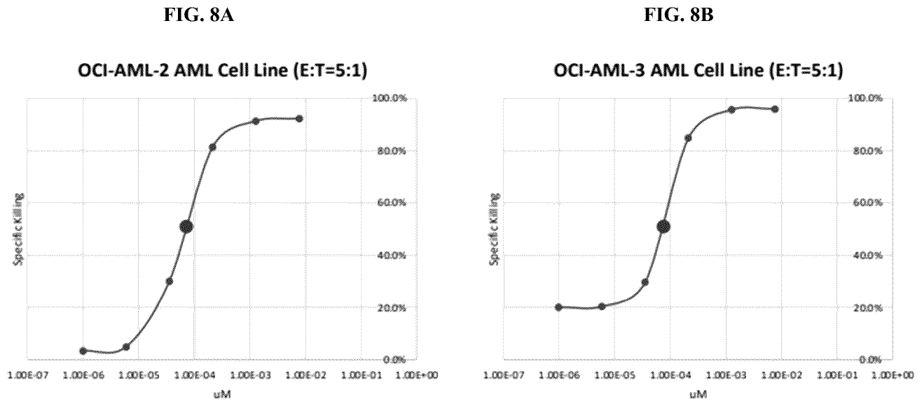

A dose titration of the CD133 BiTE is shown in FIG. 8A and FIG. 8B against HLA-A2⁺ AML cell lines OCI-AMI-2 and OCI-AMI-3, respectively. E+E cells specific for AML (1 target) were added at 5:1 E:T.

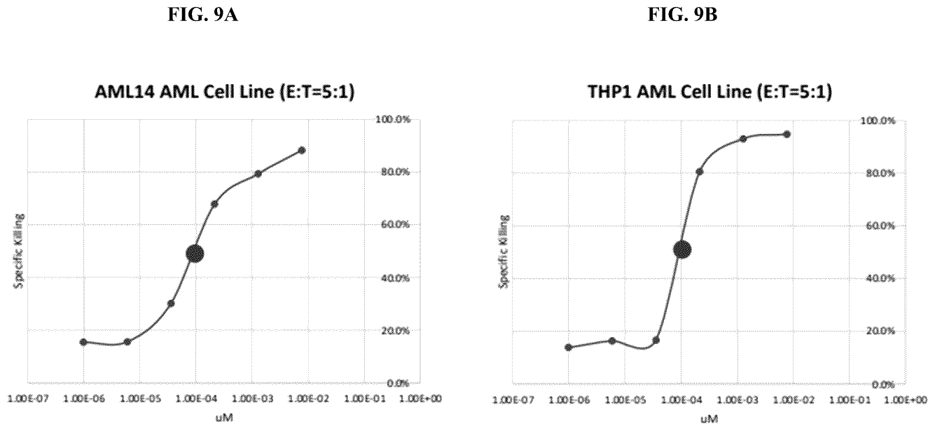

A dose titration of the CD133 BiTE is shown in FIG. 9A and FIG. 9B against HLA-A2⁺ AML cell lines AML 14 and THP1, respectively. E+E cells specific for AML (2 targets) were added at 5:1 E:T.

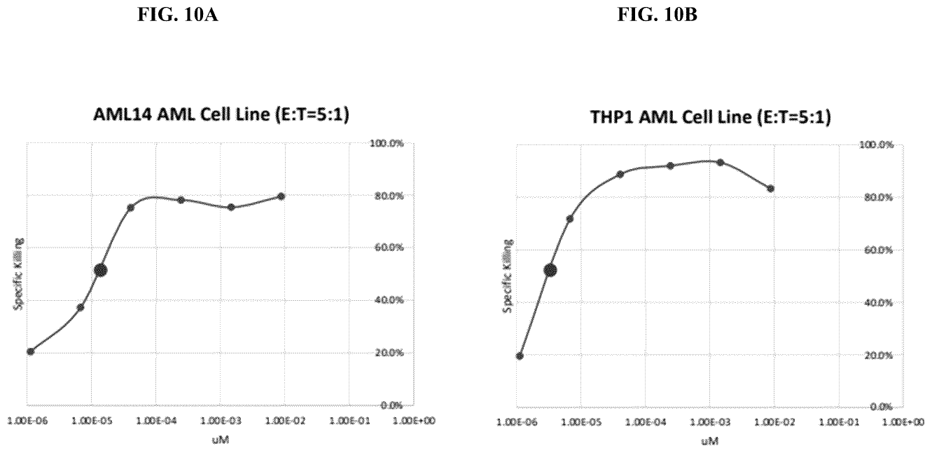

A dose titration of the Flt3 BiTE is shown in FIG. 10A and FIG. 10B against HLA-A2⁺ AML cell lines AML 14 and THP1, respectively. E+E cells specific for AML (2 targets) were added at 5:1 E:T.

Collectively, the experiments of these examples suggest that: (1) BiTEs can synergistically augment the pharmacologic activity of adoptive cell therapy, such as between 3 and 5-fold at low picomolar concentrations of the BiTE, (2) the BiTE is active at lower concentrations when combined with CTLs, and (3) that adoptive cell therapy and BiTE significantly increase both the potency and specificity of cellular killing.

### Example 3: Tumor Antigen-Specific T Cells are Synergistic with T Cell Engagers

Multiple myeloma (MM) and acute myeloid leukemia (AML) are blood cancers that remain difficult to treat because many patients have disease that does not respond to currently available therapies. Given that T cell function in MM and AML patients is often perturbed and the response to TCE therapy is determined by pre-existing endogenous T cells of the patient, this example tests, among other things, whether adoptively transferred multi-antigen specific T cells with a memory phenotype in combination with TCE therapy might improve therapy. Polyclonal cytotoxic T lymphocytes (CTL) were compared with AIM CTL. AIM CTL were primed and expanded with paramagnetic aAPCs ex vivo to provide antigen experience and specificity, essentially as described herein. The preclinical efficacy of B cell maturation antigen (BCMA)×CD3 TCE+MM-specific AIM CTL combination therapy was evaluated, as well as CD123×CD3 TCE+ AML-specific AIM CTL combination therapy both in vitro and in vivo.

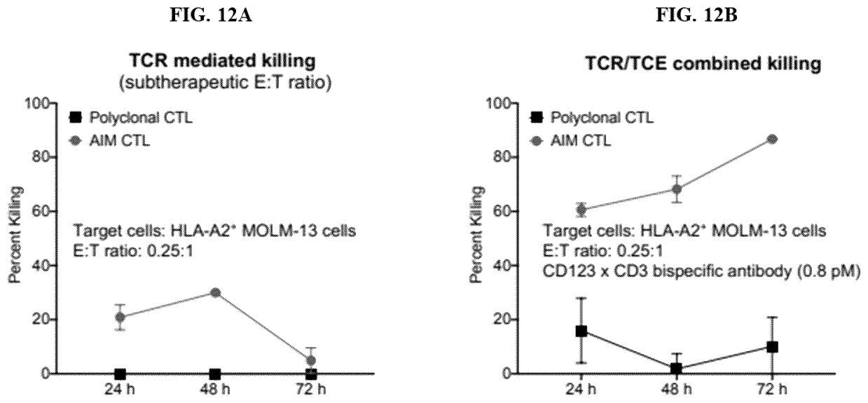

In co-culture assays with polyclonal CTL and AIM CTL, the effect of prior antigen exposure was investigated on the efficacy of the TCE against tumor cell lines by measuring the responses to T cells alone and in the presence of TCE treatment. Tumor cell survival was quantified, by determining cytotoxicity of the various culture conditions by luciferase assay. The analysis showed that, as expected, CD4 T cells had little potency. Non-HLA matched control CTL (bulk or enriched for naïve cells) without TCE showed higher cytotoxicity, but these cells were much less potent than AIM CTL (FIG. 12A). Combined TCR and TCE-mediated target cell killing was most efficient when using AIM CTL as effector cells (FIG. 12B). Correcting the data for TCR-mediated killing revealed that antigen-experienced AIM CTL were indeed superior effectors of TCE potency than antigen inexperienced control CTL. This finding was reproducible regardless of the HLA type of the target cells, consistent with the TCR-independent mechanism of action of TCE-mediated killing.

Nuclear factor of activated T cells (NFAT) transcriptional reporter assays with Jurkat effector cells and AML target cells revealed that TCE treatment decreased NFAT transcriptional activity in TCE effector cells; moreover, TCE treatment in vivo (treating mice with AIM CTL+/−TCE) increased CD3 levels on AIM CTL. Both observations are consistent with reduced TCR-mediated T cell activation in the context of TCE therapy. Furthermore, BCMA×CD3 TCE treatment of mice in a MM xenograft model resulted in decreased PD-1 expression on AIM CTL and enhanced the in vivo efficacy of AIM CTL. These studies show that AIM CTL display strong anti-tumor activity, and even subtherapeutic AIM CTL cell doses can be highly efficacious when armed with a TCE. Importantly, these data indicate that AIM CTL cells are optimal TCE effectors, superior to less experienced, polyclonal CTL (such as the patient's endogenous T cells). Combining AIM CTL with TCE therapy has the additional benefits of complementary targets and mechanisms of action, reducing the risk of immune escape. Moreover, these studies suggest that TCE treatment reduces NFAT transcriptional activity and PD-1 induction associated with physiological TCR-mediated T cell activation. This suggests that TCE-mediated T cell activation can partially suppress TCR-mediated T cell activation in AIM CTL, resulting in excellent tumor-specific cytolytic activity while inducing a transcriptional signature that preserves T cell fitness. Following withdrawal of TCE therapy, less exhausted AIM CTL will be present, facilitating the persistence of long-lasting anti-tumor activity and immunosurveillance.

Combination of AIM CTL (autologous or HLA matched allogeneic) with TCE therapy is therefore a highly promising and innovative strategy for the induction of durable antitumor activity, and this approach has potential for clinical translation as a novel combinatorial immunotherapy.

1. A method for treating a subject in need of a T cell therapy, comprising administering a cytotoxic T lymphocyte (CTL) therapy to the subject, and administering one or more bispecific T cell engagers (BiTE) at an amount effective to potentiate the CTL therapy.

2.-3. (canceled)

4. The method of claim 1, wherein the CTLs are activated and/or expanded ex vivo from peripheral blood or tumor infiltrating lymphocytes, optionally using artificial antigen presenting cells presenting one or more target antigens.

5. The method of claim 4, wherein greater than about 50% of the CTLs comprise a memory phenotype; or greater than about 75%, or greater than about 85%, or greater than about 95% of the CTLs have a memory phenotype.

6. (canceled)

7. The method of claim 1, wherein the CTLs comprise central memory and effector memory T cells.

8.-14. (canceled)

15. The method of claim 1, wherein the CTLs are administered at a dose of from about 10⁶ to about 10¹⁰ cells per administration.

16. The method of claim 1, wherein the CTLs are administered at a dose of less than about 10⁹ cells per administration.

17.-18. (canceled)

19. The method of claim 1, wherein the BiTE is administered as a polypeptide composition or is administered as a BiTE-encoding polynucleotide, optionally wherein the BiTE-encoding polynucleotide is introduced to the CTLs ex vivo.

20. The method of claim 1, wherein the BiTE comprises a first domain that binds a T cell surface antigen, and a second domain that binds a target cell antigen.

21. The method of claim 20, wherein the BiTE is a bispecific antibody, dimerized natural ligand(s) or portions thereof, or comprises one or more single chain antibodies (scFv) that are dimerized.

22. The method of claim 19, wherein the first domain binds CD3.

23. The method of claim 19, wherein the first domain is an HLA-target peptide complex.

24. The method of claim 23, wherein one or more peptide antigens are selected from WT1, PRAME, Survivin, CyclinA1, XBP1, CD138, CS1, NY-ESO1, SOX2, EBV, CMV, RHAMM, PR3, MART-1/MELAN-A, and gp100.

25.-27. (canceled)

28. The method of claim 1, wherein the subject has a cancer.

29. The method of claim 28, wherein the cancer is a hematological malignancy, optionally selected from acute myeloid leukemia, chronic myelogenous leukemia, childhood acute leukemia, non-Hodgkin's lymphomas, acute lymphocytic leukemia, chronic lymphocytic leukemia, myelodysplastic syndrome, malignant cutaneous T-cells, mycosis fungoids, non-MF cutaneous T-cell lymphoma, lymphomatoid papulosis, and T-cell rich cutaneous lymphoid hyperplasia.

30. The method of claim 29, wherein the second domain binds a target expressed on the surface of cancer cells.

31. The method of claim 30, wherein the second domain binds CD19.

32. The method of claim 30, wherein the second domain binds a tumor target selected from BCMA, CD20, GPRC5D, FcRH5, CD38, gpA33, CD33, CD123, CLE12A, Flt3, and CD79b.

33.-35. (canceled)

36. The method of claim 28, wherein the cancer is a solid tumor, optionally selected from a carcinoma or sarcoma.

37.-38. (canceled)

39. The method of claim 28, wherein the BiTE is selected from blinatumomab, catumaxomab, mosunetuzumab, elranatamab, epcoritamab, glofitamab, talquetamab, teclistamab, flotetuzumab, odronextamab, plamotamab, tarlatamab, vibecotamab, acapatamab, cevostamab, emfizatamab, linvoseltamab, nivatrotamab, tidutamab, and ubamatamab.

40.-50. (canceled)

51. A method for treating a subject in need of T cell therapy, comprising: administering an antigen presenting composition to the subject, thereby activating T cells in the subject in vivo, and administering one or more bispecific T cell engagers (BiTE).

52.-92. (canceled)

93. A composition comprising a cytotoxic T lymphocyte (CTL) therapy and one or more bispecific T cell engagers (BiTE), and a pharmaceutically-acceptable vehicle.

94.-130. (canceled)

131. A composition comprising an mRNA encoding a BiTE encapsulated by or conjugated to a nanoparticle, wherein the nanoparticle is optionally an antigen presenting composition.

132.-154. (canceled)
